SEQUENCE LISTING (14 sequences)

SEQ ID NO: 1 - protein, 7 aa, Artificial Sequence
  Description of Artificial Sequence Synthetic metal affinity tag
HNHRHKH

SEQ ID NO: 2 - protein, 8 aa, Artificial Sequence
  Description of Artificial Sequence Synthetic FLAG peptide
DYKDDDDK

SEQ ID NO: 3 - protein, 13 aa, Artificial Sequence
  Description of Artificial Sequence Synthetic peptide
SAVRAXSSVPGVR

SEQ ID NO: 4 - protein, 13 aa, Artificial Sequence
  Description of Artificial Sequence Synthetic peptide
SAVRLRSSVPGVR

SEQ ID NO: 5 - protein, 10 aa, Artificial Sequence
  Description of Artificial Sequence Synthetic peptide
FWADKEEEWR

SEQ ID NO: 6 - protein, 8 aa, Artificial Sequence
  Description of Artificial Sequence Synthetic His tag
HHHHHHHH

SEQ ID NO: 7 - protein, 20 aa, Artificial Sequence
  Description of Artificial Sequence Synthetic peptide
DGLAHLDNLKGTFATLSELH

SEQ ID NO: 8 - protein, 22 aa, Artificial Sequence
  Description of Artificial Sequence Synthetic peptide
EWMGGIIPIFGTANYAQKFRGR

SEQ ID NO: 9 - protein, 15 aa, Artificial Sequence
  Description of Artificial Sequence Synthetic peptide
CPKEIPKGSKNTEVL

SEQ ID NO: 10 - protein, 13 aa, Artificial Sequence
  Description of Artificial Sequence Synthetic peptide
SAVRARSSVPGVR

SEQ ID NO: 11 - protein, 14 aa, Artificial Sequence [sequence omitted]

SEQ ID NO: 12 - protein, 14 aa, Artificial Sequence
  Description of Artificial Sequence Synthetic peptide
TGSSTGGXQGSHHE

SEQ ID NO: 13 - protein, 9 aa, Artificial Sequence
  Description of Artificial Sequence Synthetic peptide
AXSSVPGVR

SEQ ID NO: 14 - protein, 13 aa, Artificial Sequence
  Description of Artificial Sequence Synthetic peptide
SAVQAXSSVPGVR

(19) United States
(12) Patent Application Publication
(10) Pub. No.: US 2011/0165606 A1
(43) Pub. Date: Jul. 7, 2011

(54) METHODS FOR MODIFYING, ISOLATING, DETECTING, VISUALIZING, AND QUANTIFYING CITRULLINATED AND/OR HOMOCITRULLINATED PEPTIDES, POLYPEPTIDES AND PROTEINS

(76) Inventors: Astrid E.V. Tutturen, Oslo (NO); Anders Holm, Blommenholm (NO); Burkhard Fleckenstein, Oslo (NO)

(21) Appl. No.: 12/664,494

(22) PCT Filed: Jun. 12, 2008
(86) PCT No.: PCT/IB08/02407

§ 371 Date: Jun. 4, 2010

Related U.S. Application Data
(60) Provisional application No. 60/934,718, filed on Jun. 15, 2007.

(51) Int. Cl. C12Q 1/44; C12Q 1/34; C12Q 1/28; G01N 33/00; C07K 2/00; C07K 1/02; C07K 14/00; C07D 495/04; C07D 311/82; C07C 233/77; C08G 63/91

(52) U.S. Cl. 435/19; 435/18; 435/28; 436/86; 530/300; 530/345; 530/350; 530/402; 548/304.1; 549/388; 564/169; 525/450

(57) ABSTRACT

The present invention relates to methods and compositions for modifying, isolating, detecting, visualizing, and quantifying citrulline and/or homocitrulline-containing peptides, polypeptides, and proteins using mono- and disubstituted glyoxal derivatives. The invention also provides kits for modifying, isolating, detecting, visualizing, and quantifying the relative amounts of citrulline and/or homocitrulline-containing peptides, polypeptides, or proteins in solutions or samples.

### RELATED APPLICATIONS

This application claims priority to U.S. Patent Application No. 60/934,718 filed on Jun. 15, 2007, which is incorporated herein by reference in its entirety.

### FIELD OF THE INVENTION

The present invention relates to methods of studying posttranslationally modified proteins and peptides and compositions used for such studies. The present invention further relates to methods and compositions for specifically modifying, isolating, detecting, visualizing, and quantifying citrulline and/or homocitrulline-containing proteins and peptides using mono- and disubstituted glyoxal derivatives.

### BACKGROUND OF THE INVENTION

Nearly all proteins undergo some form of posttranslational modification. Such modifications alter specific amino acid residues of a protein after translation, tremendously increasing the structural and functional diversity of the proteome. Common posttranslational modifications include, but are not limited to, the addition of phosphate groups (phosphorylation), methyl groups (methylation), sugar molecules (glycosylation), and other chemical changes to specific amino acids, such as citrullination. These modifications frequently have significant effects on the structure, function, and biological activity of the modified protein. György, B. et al., “Citrullination: A posttranslational modification in health and disease,” INT'L J. BIOCHEM. CELL BIOL. 38:1662-77 (2006).

Citrulline is a non-standard amino acid not used in protein synthesis. Vossenaar, E. R., et al., “PAD, a growing family of citrullinating enzymes: genes, features and involvement in disease,” BIOESSAYS 25:1106-1118 (2003). Citrullination of proteins and peptides results from the dei ination of peptide-bound arginine residues. Deimination of the arginine amino acid is depicted in FIG. 1A. Deimination is catalyzed by a family of Ca²⁺-dependent enzymes called peptidylarginine deiminases (“PADs”), of which there are five known mammalian isoforms. Vossenaar, E. R. et al., at 1107-1112. One important consequence of the conversion of arginine to citrulline is the loss of a positive charge, because arginine is protonated under physiological conditions, while citrulline is not charged.

Citrullinated proteins are thought to play an essential role in numerous physiological processes including terminal differentiation of the epidermis and apoptosis, and more generally in the regulation of transcription. György, B. et al., at 1666-1667; Arita, K. et al., “Structural basis for histone N-terminal recognition by human peptidylarginine deiminase 4,” PROC. NATL. ACAD. SCI. U.S.A. 103(14):5291-5296 (2006). One PAD isoform, PAD4, is present in the nucleus. Recent studies have shown that histones H3 and H4 are citrullinated at their amino termini, suggesting that citrullination may also play a role in gene transcription, either alone, or as part of the dynamic “histone code.” Arita, K. et al., at 5291.

Citrullinated proteins have also been implicated in the pathogenesis of autoimmune disorders such as rheumatoid arthritis (“RA”) and multiple sclerosis (“MS”). György, B. et al., at 1669-72. RA is a chronic autoimmune disease characterized by symmetric inflammation of the peripheral synovial joints. György, B. et al., at 1669. At early stages of the disease, the inflamed joints are painful and swollen, but later, if left untreated, the inflammation may lead to destruction of cartilage and bone in the affected joints. György, B. et al., at 1669. Autoantibodies recognizing citrullinated proteins with high sensitivity and specificity are present in serum obtained from RA patients. Such antibodies are widely used as diagnostic markers of the disease in clinical practice. Nevertheless, the citrullinated antigens responsible for the onset of RA have not yet been isolated or characterized.

MS is a severe autoimmune disease that affects myelin sheaths of neurons in the central nervous system (“CNS”). As the disease progresses, neurons of the CNS gradually lose their myelin sheath synthesized by oligodendroglial cells. György, B. et al., at 1672. This demyelination interferes with the ability of the neurons to conduct nerve impulses, eventually causing paralysis and death. György, B. et al., at 1672. Myelin basic protein (“MBP”) is one of the major protein components of the myelin sheath. Studies have shown that MS may be caused by overcitrullination of MBP, indicated both by an increase in the ratio of citrullinated MBP to total MBP, and in the total number of citrullines within MBP. György, B. et al., at 1672.

Homocitrulline is also a non-coded amino acid that is formed by carbamylation of lysine residues in a protein or peptide. Urea exists in serum in equilibrium with trace amounts of cyanate (OCN⁻). Wang, Z. et al., “Protein Carbamylation links inflammation, smoking, uremia and atherogenesis,” NATURE MEDICINE 13:1176-1184 (2007). Carbamylation can occur by reaction of cyanate with the terminal amine of a lysine residue to form a homocitrulline residue, as depicted in FIG. 1B. Carbamylation can be catalyzed by the leukocye heme peroxidase MPO and is involved in pathways linked to inflammation, uremia and arthrosclerosis. Wang, Z. et al., at 1182. The degree of homocitrullinated proteins may serve as a gauge of atherosclerotic coronary artery disease, which can be useful in patients who smoke. Wang, Z. et al., at 1182.

Citrullinated proteins are involved in the pathogenesis of two relatively well-known autoimmune disorders, while homocitrullinated proteins play a role in coronary and inflammation conditions. Both citrulline and homocitrulline have a ureido substituent, such that they both can be modified by the same conditions and reagents that react with a ureido group. However, the citrullinated and homocitrullinated proteomes and peptidomes have not been well-characterized. Thus, methods are needed to identify and characterize citrullinated and/or homocitrullinated peptides, polypeptides, and proteins.

It is accordingly an object of the invention to provide novel, specific methods for the chemical modification of citrulline and homocitrulline residues in peptides, polypeptides, and proteins, as well as various compositions for use in conjunction with the methods of the invention. It is also an object of the invention to provide methods of isolating, detecting, visualizing and quantifying citrulline and/or homocitrulline-containing peptides, polypeptides, and proteins.

### SUMMARY OF THE INVENTION

It is one of the objects of the invention to provide methods and compositions for specifically modifying, isolating, detecting, visualizing, and quantifying citrulline and/or homocitrulline-containing peptides, polypeptides, and proteins using mono- and disubstituted glyoxal derivatives. Thus, in certain embodiments, compounds of formula (I) are provided:

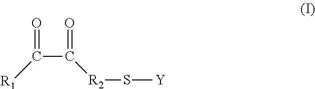

wherein R₁ and R₂ comprise any branched or unbranched alkyl or aryl chain of different size, length, hydrophobicity, water-solubility, positive or negative inductive effect, positive or negative mesomeric effect, or a hydrogen; wherein S is a spacer comprising any branched or unbranched aliphatic or polyethylene glycol-based chain of variable size, length, and hydrophobicity; and wherein Y is a physical or molecular tag that facilitates identification, visualization, detection or purification of citrulline and/or homocitrulline-containing peptides, polypeptides or proteins labeled with the compound. In certain embodiments, R₁ is selected from the group consisting of —H, —CH₃, —CH₂CH₃, and a phenyl group. In certain embodiments, R₂ is selected from the group consisting of —H, —CH₃, —CH₂CH₃, and a phenyl group. In certain embodiments, the spacer S comprises —(CH₂)ₙ— or —(O—CH₂—CH₂)ₙ—. In certain embodiments, n=1 to 25. In certain embodiments, the spacer S further comprises a cleavage site, a disulfide bond, a photocleavable group, a base labile group, or an enzymatic cleavage site. In certain embodiments, the photocleavable group is o-nitrobenzyl or pivaloyl. In certain embodiments, the enzymatic cleavage site is for a commercially available protease. In certain embodiments, the commercially available protease is trypsin, chymotrypsin, Lys-C, Asp-N, or Glu-C. In certain embodiments, Y is a magnetic bead, resin, or a solid support. In certain other embodiments, Y is selected from the group consisting of biotin, iminobiotin, biotinyl-6-aminohexanoic acid, a His-tag, a metal affinity tag (SEQ ID NO:1), a FLAG peptide (SEQ ID NO:2), digoxin, a dinitrophenyl group, a nitrotyrosine residue, fluorescein isothiocyanate, Texas Red, and rhodamine.

In certain embodiments, methods of modifying citrulline and/or homocitrulline-containing peptides, polypeptides, or proteins under acidic conditions are provided, comprising contacting a solution or sample comprising at least one citrulline and/or homocitrulline-containing peptide, polypeptide or protein, and at least one citrulline and/or homocitrulline-reactive compound of formula (I) as described in paragraph [010] above, wherein the citrulline and/or homocitrulline-reactive compound of formula (I) becomes covalently attached to at least one citrulline and/or homocitrulline residue within the at least one citrulline and/or homocitrulline-containing peptide, polypeptide, or protein in the solution or sample. In certain embodiments, the solution or sample and the at least one citrulline and/or homocitrulline-reactive compound of formula (I) are incubated at a temperature between 4° C. and 70° C. for 1 to 24 hours. In one embodiment, the peptide, polypeptide or protein comprises citrulline. In another embodiment, the peptide, polypeptide or protein comprises homocitrulline. In certain embodiments, the sample is a biological sample. In certain other embodiments, the biological sample is selected from a tissue biopsy, cultured cells, bacterial or viral cultures, cerebrospinal fluid, serum, blood, plasma, saliva, amniotic fluid, synovial fluid, lacrimal fluid or tears, milk, lymph, urine, and sweat. In one embodiment, the biological sample is synovial fluid.

In certain embodiments, methods of isolating, enriching, or purifying citrulline and/or homocitrulline-containing peptides, polypeptides, or proteins from a solution or sample are provided, comprising contacting at least one citrulline and/or homocitrulline-reactive compound of formula (I) as described in paragraph [010] above, with a solution or sample containing at least one citrulline and/or homocitrulline-containing peptide, polypeptide, or protein, wherein the citrulline and/or homocitrulline-reactive compound of formula (I) becomes covalently attached to at least one citrulline and/or homocitrulline residue within the at least one citrulline and/or homocitrulline-containing peptide, polypeptide, or protein in the solution or sample; collecting the modified citrulline and/or homocitrulline-containing peptides, polypeptides, or proteins; and removing the unmodified peptides, polypeptides, or proteins from the solution or sample. In certain embodiments, the citrulline and/or homocitrulline-reactive compound is Biotin-PEG-4-glyoxalbenzoic acid (Biotin-PEG-GBA). In certain embodiments, the solution or sample and the at least one citrulline and/or homocitrulline-reactive compound of formula (I) are incubated at a temperature between 4° C. and 70° C. for 1 to 24 hours. In one embodiment, the peptide, polypeptide or protein comprises citrulline. In another embodiment, the peptide, polypeptide or protein comprises homocitrulline. In certain embodiments, the sample is a biological sample. In certain other embodiments, the biological sample is selected from a tissue biopsy, cultured cells, bacterial or viral cultures, cerebrospinal fluid, serum, blood, plasma, saliva, amniotic fluid, synovial fluid, lacrimal fluid or tears, milk, lymph, urine, and sweat. In one embodiment, the biological sample is synovial fluid.

In certain embodiments, methods of isolating, enriching, or purifying citrulline and/or homocitrulline-containing peptides, polypeptides, or proteins from a solution or sample are provided, comprising preparing a solution or sample comprising at least one citrulline and/or homocitrulline-containing peptide, polypeptide, or protein; contacting the solution or sample with resin or beads under acidic conditions, wherein the resin or beads comprise a citrulline and/or homocitrulline-reactive group linked to the resin or beads by a spacer which may be cleavable; allowing the citrulline and/or homocitrulline-reactive group on the resin or beads to react with and modify the at least one citrulline and/or homocitrulline-containing peptide, polypeptide, or protein in the solution or sample; removing any non-citrulline and/or homocitrulline-containing peptides, polypeptides, or proteins from the solution or sample; cleaving the spacer; and collecting the modified at least one citrulline and/or homocitrulline-containing peptide, polypeptide, or protein. In one embodiment, the peptide, polypeptide or protein comprises citrulline. In another embodiment, the peptide, polypeptide or protein comprises homocitrulline. In certain embodiments, the sample is a biological sample. In certain embodiments, the biological sample is selected from a tissue biopsy, cultured cells, bacterial or viral cultures, cerebrospinal fluid, serum, blood, plasma, saliva, amniotic fluid, synovial fluid, lacrimal fluid or tears, milk, lymph, urine, and sweat. In one embodiment, the biological sample is synovial fluid. In certain embodiments, the resin comprising a cleavable spacer is TentaGel S 4-hydroxymethylbenzoic acid resin. In certain embodiments, the beads comprising a cleavable spacer are M-280 4-hydroxymethylbenzoic acid Dynabeads®. In certain embodiments, the resin is a sarcosine dimethylacrylamide resin (PL-DMA-resin).

In certain embodiments, methods of detecting citrulline and/or homocitrulline-containing peptides, polypeptides, or proteins in a solution or sample are provided, comprising obtaining a solution or sample comprising at least one citrulline and/or homocitrulline-containing peptide, polypeptide, or protein; modifying the at least one citrulline and/or homocitrulline-containing peptide, polypeptide, or protein in the solution or sample by the method described in paragraph [011] above; and detecting the modified citrulline and/or homocitrulline-containing peptide, polypeptide, or protein in the solution or sample. In one embodiment, the peptide, polypeptide or protein comprises citrulline. In another embodiment, the peptide, polypeptide or protein comprises homocitrulline. In certain embodiments, the sample is a biological sample. In certain embodiments, the biological sample is selected from a tissue biopsy, cultured cells, bacterial or viral cultures, cerebrospinal fluid, serum, blood, plasma, saliva, amniotic fluid, synovial fluid, lacrimal fluid or tears, milk, lymph, urine, and sweat. In one embodiment, the biological sample is synovial fluid.

In certain embodiments, the method further comprises the step of fractionating or fixing the biological sample before detecting the modified citrulline and/or homocitrulline-containing peptide, polypeptide, or protein. In certain embodiments, the fractionating of the biological sample is by chromatography, filtration, precipitation (e.g., by salt, pH, or organic solvent) or gel electrophoresis and Western blotting. In certain other embodiments, the fixation of the biological sample is by formalin fixation, paraffin embedding, and sectioning.

In certain embodiments, methods of quantifying relative amounts of citrulline and/or homocitrulline-containing peptides, polypeptides, or proteins in a solution or sample are provided, comprising (a) obtaining a first solution or sample comprising at least one citrulline and/or homocitrulline-containing peptide, polypeptide, or protein, and a second solution or sample comprising at least one citrulline and/or homocitrulline-containing peptide, polypeptide, or protein; (b) modifying the at least one citrulline and/or homocitrulline-containing peptide, polypeptide, or protein in the first solution or sample with a ¹²C-labeled version of a citrulline and/or homocitrulline-reactive compound of formula (I); (c) modifying the at least one citrulline and/or homocitrulline-containing peptide, polypeptide, or protein in the second solution or sample with a ¹³C-labeled version of a citrulline and/or homocitrulline-reactive compound of foimula (I); (d) mixing the first solution or sample modified with the ¹²C-labeled version of a citrulline and/or homocitrulline-reactive compound and the second solution or samples modified with the ¹³C-labeled version of a citrulline and/or homocitrulline-reactive compound together; (e) digesting the mixture with trypsin; (f) collecting the modified citrulline and/or homocitrulline-containing peptides, polypeptides, or proteins from the trypsin-digested mixture; (g) determining the relative amount of citrulline and/or homocitrulline-containing peptide, polypeptide, or protein collected from the first and second biological samples by MALDI-TOF or LC-tandem mass spectrometry; and (h) identifying each modified peptide, polypeptide, or protein by MS/MS. In certain embodiments, the citrulline and/or homocitrulline-reactive compound of formula (I) is Biotin-PEG-GBA. In one embodiment, the peptide, polypeptide or protein comprises citrulline. In another embodiment, the peptide, polypeptide or protein comprises homocitrulline. In certain embodiments, the sample is a biological sample. In certain embodiments, the biological sample is selected from a tissue biopsy, cultured cells, bacterial or viral cultures, cerebrospinal fluid, serum, blood, plasma, saliva, amniotic fluid, synovial fluid, lacrimal fluid or tears, milk, lymph, urine, and sweat. In one embodiment, the biological sample is synovial fluid.

In certain embodiments, methods of assessing the efficacy of a treatment for a disease associated with altered citrullination and/or homocitrullination of at least one protein are provided, comprising: (a) obtaining a first biological sample from a patient suffering from the disease before treatment; (b) administering the treatment; (c) obtaining a second biological sample from said patient after treatment; and (d) determining the relative amounts of the at least one citrulline and/or homocitrulline-containing peptide, polypeptide, or protein in the biological samples by the methods described in paragraph [016] above, wherein altered citrullination and/or homocitrullination of at least one peptide, polypeptide, or protein in the second biological sample relative to the first biological sample indicates the treatment is effective. In certain embodiments, the biological sample is selected from a tissue biopsy, cultured cells, bacterial or viral cultures, cerebrospinal fluid, serum, blood, plasma, saliva, amniotic fluid, synovial fluid, lacrimal fluid or tears, milk, lymph, urine, and sweat. In one embodiment, the biological sample is synovial fluid. In certain embodiments, the disease is multiple sclerosis. In certain embodiments, the disease is rheumatoid arthritis.

In certain embodiments, kits for quantifying the relative amounts of citrulline and/or homocitrulline-containing peptides, polypeptides, or proteins in a solution or sample by the methods described in paragraph [016] above are provided, comprising (a) a light and a heavy version of a citrulline and/or homocitrulline-reactive compound of formula (I), (b) avidin/streptavidin coated magnetic beads; and (c) tubes, containers, reaction vessels, buffers, and reagents required to perform the steps of the method. In certain embodiments, the citrulline and/or homocitrulline reactive group may be biotinylated. In certain embodiments, the citrulline and/or homocitrulline-reactive compound of formula (I) is Biotin-PEG-GBA.

In certain embodiments, kits for isolating, enriching, or purifying citrulline and/or homocitrulline-containing peptides, polypeptides, or proteins from a solution or sample are provided, comprising (a) beads, resins or solid supports derivatized with a citrulline and/or homocitrulline-reactive compound of formula (I); and (b) tubes, containers, reaction vessels, buffers, and reagents required to isolate, enrich, or purify citrulline and/or homocitrulline-containing peptides, polypeptides, or proteins. In certain embodiments, the citrulline and/or homocitrulline-reactive compound of formula (I) is GBA.

In certain embodiments, kits for detecting or visualizing citrulline and/or homocitrulline-containing peptides, polypeptides, or proteins in a solution or sample are provided, comprising (a) a citrulline and/or homocitrulline-reactive compound of formula (I); and (b) tubes, containers, reaction vessels, buffers, and reagents required to detect or visualize citrulline and/or homocitrulline-containing peptides, polypeptides, or proteins in a biological sample. In certain embodiments, the citrulline and/or homocitrulline-reactive compound of formula (I) is Biotin-PEG-GBA, and the citrulline and/or homocitrulline-containing peptides, polypeptides, and proteins are visualized using streptavidin conjugated with alkaline phosphatase or horseradish peroxidase by standard methods. In certain embodiments, the solution or sample undergoes additional fractionation or fixation. In certain embodiments, the additional fractionation is by chromatography, filtration, precipitation by salt, pH, or organic solvent, gel electrophoresis, or Western blotting. In certain other embodiments, the fixation of the solution or sample is by formalin fixation, paraffin embedding, and sectioning.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1A depicts the conversion of arginine to citrulline by peptidylarginine deiminase (PAD). In vivo, PAD deiminates backbone-bound arginine residues. FIG. 1B depicts the conversion of lysine to homocitrulline by carbamylation.

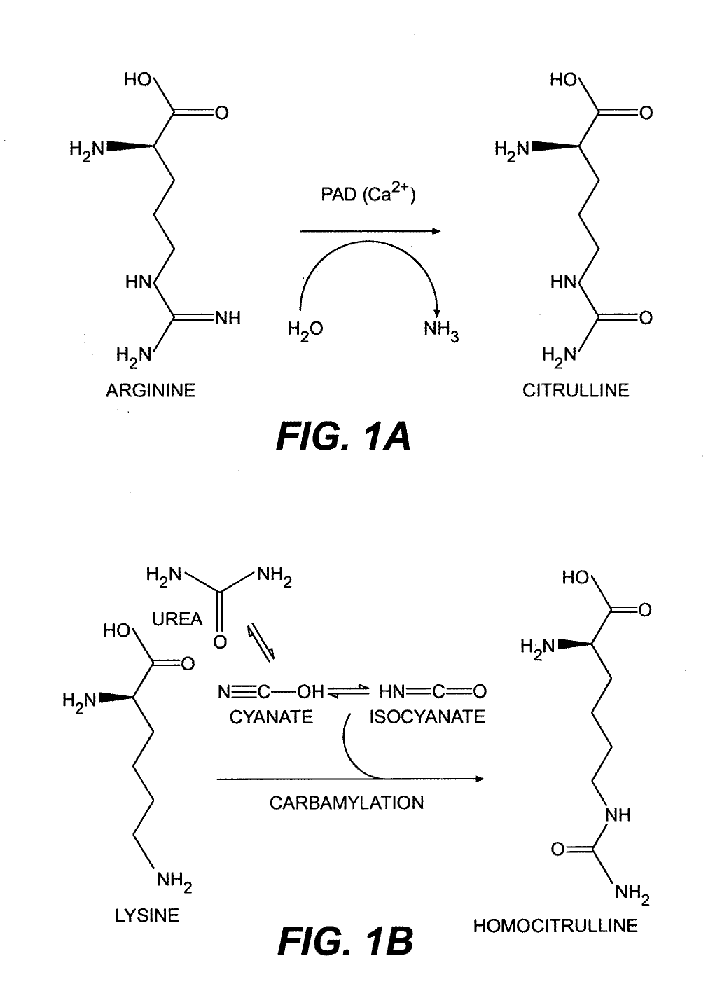

FIG. 2 depicts the modification of citrulline and homocitrulline by 4-glyoxalbenzoic acid (GBA), with R indicating the HOOC-phenyl moiety.

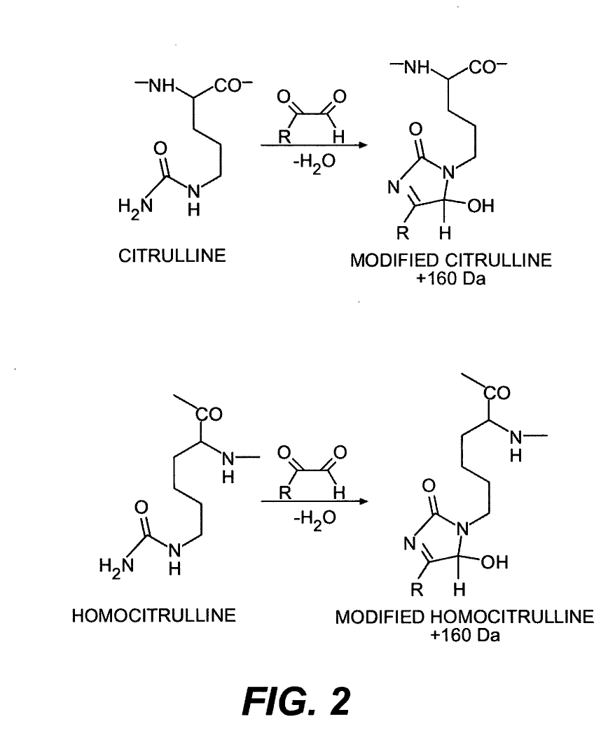

FIG. 3 depicts the MALDI-TOF MS spectrum of the polypeptide SAVRA-Cit-SSVPGVR before (top) and after (bottom) modification with 2,3-butanedione and D-biotin, as described in Example 1.

FIG. 4 depicts the MALDI-TOF MS spectrum of a mixture of the unmodified polypeptide SAVRA-Cit-SSVPGVR with SAVRA-Cit-SSVPGVR biotinylated using 2,3-butanedione and D-biotin (top) and of the supernatant obtained after depletion of that mixture with streptavidin-coated beads (bottom), as described in Example 1.

FIG. 5 depicts the MALDI-TOF MS spectrum of the unmodified SAVRA-Cit-SSVPGVR polypeptide (top), the SAVRA-Cit-SSVPGVR polypeptide modified with methylglyoxal (middle), and the SAVRA-Cit-SSVPGVR polypeptide modified with phenylglyoxal (bottom), all as described in Example 1.

FIG. 6 depicts a method of synthesizing 4-glyoxalbenzoic acid (GBA) by oxidizing 4-acetylbenzoic acid with selenium dioxide as described in Example 1.

FIG. 7a depicts the general formula of monosubstituted glyoxal derivatives. FIG. 7b depicts the structure of 4-oxopentanoic acid and 3,3-dimethyl-4-oxopentanoic acid.

FIG. 8 depicts the MALDI-TOF MS spectrum of unmodified SAVRA-Cit-SSVPGVR polypeptide (m/z 1342.776, top), and of SAVRA-Cit-SSVPGVR polypeptide modified with GBA synthesized by the protocol described in Example 2 (m/z 1502.953, bottom).

FIG. 9A depicts the structure of a GBA-PEG-biotin derivative prepared by the first procedure of Example 2. FIG. 9B depicts the structure of a GBA-PEG-biotin derivative prepared by the alternative procedure of Example 2.

FIG. 10A depicts the MALDI-TOF MS spectrum of a simple peptide mixture containing the peptides SAVRA-Cit-SSVPGVR (m/z 1342.711) and SAVRL-R-SSVPGVR (m/z 1382.790), and SAVRA-Cit-SSVPGVR modified with the Biotin-PEG-GBA (m/z 1930.946) synthesized by the first procedure of Example 2. FIG. 10B depicts the SAVRA-Cit-SSVPGVR (m/z 1342.77) peptide before and after modification with Biotin-PEG-GBA (m/z 1858.98) synthesized according to the alternative procedure of Example 2.

FIG. 11 depicts the MALDI-TOF MS spectrum of the flow-through of the sample from FIG. 10 when applied to a monomeric avidin column showing that the modified SAVRA-Cit-SSVPGVR (m/z 1930.9) is almost completely retained on the column, while unmodified SAVRA-Cit-SSVPGVR and SAVRL-R-SSVPGVR were washed through the column.

FIG. 12 depicts the MALDI-TOF MS spectrum of the supernatant following treatment of the beads with 0.3 M NaOH, showing a prominent peak at m/z 1502.789 corresponding to SAVRA-Cit-SSVPGVR modified with GBA, and a series of smaller peaks characteristic of PEG polymers, as discussed in Example 3.

FIG. 13 depicts the SDS-PAGE separation of BSA, lysozyme and albumin (left) and the Western Blot of citrullinated BSA (right), as discussed in Example 9.

FIG. 14 depicts the MALDI-TOF MS spectra of the lower band (top) and the upper bands (bottom) of the Western blot, as discussed in Example 9.

FIG. 15 depicts the MALDI TOF/TOF MS spectrum of SAVRA-Cit-SSVPGVR peptide after modification by Biotin-PEG-GBA to determine the position of the citrulline residue, as discussed in Example 2.

FIG. 16 depicts the MALDI-TOF MS spectra of a sample of Gel section 4 prior to modification (top) and its streptavidin eluate (bottom), as discussed in Example 11.

FIG. 17 depicts the MALDI-TOF MS spectra of a sample of Gel section 8 prior to modification (top) and its streptavidin eluate (bottom), as discussed in Example 11.

FIG. 18 depicts the structure of PL-DMA/HMB/GBA as discussed in Example 12.

FIG. 19 depicts the MALDI-TOF MS spectra of a BSA digest before and after modification with GBA, as discussed in Example 12.

FIG. 20 depicts the MALDI-TOF MS spectra of a deiminated BSA tryptic digest before and after modification with GBA, as discussed in Example 12.

FIG. 21A depicts the MALDI-TOF MS spectrum of peptide Ac-FWADKEEEWR. FIG. 21B depicts the MALDI-TOF MS spectrum of carbamylated Ac-FWADKEEEWR. FIG. 21C depicts the MALDI-TOF MS spectrum of carbamylated Ac-FWADKEEEWR modified by Biotin-PEG-GBA, as discussed in Example 13.

### BRIEF DESCRIPTION OF THE SEQUENCES

SEQ ID NO:1 is the amino acid sequence of the metal affinity tag (“MAT”).

SEQ ID NO:2 is the amino acid sequence of the FLAG peptide.

### DESCRIPTION OF EXEMPLARY EMBODIMENTS

Reference will now be made in detail to exemplary embodiments of the invention, examples of which are illustrated in the accompanying drawings.

### DEFINITIONS

The term “biological sample,” as used herein, means any biological material collected from cells, tissues, or organs of a subject. The term also encompasses peptides, polypeptides, or proteins prepared or produced by commonly used recombinant or synthetic methods. The source of the biological sample may vary depending on the particular symptoms present in the subject to be diagnosed. The biological sample may be analyzed immediately after it is obtained, or stored. If stored, the sample may be equilibrated with an appropriate storage buffer, and kept at 4° C., at −20° C., at −70° C., or even in cryogenic liquids, such as liquid nitrogen or liquid helium. In certain embodiments, the biological sample may consist of blood, serum, or plasma. In certain other embodiments, the biological sample may consist of amniotic fluid or milk. In other embodiments, the biological sample may consist of a biopsy or tissue sample, cultured cells, or a cell suspension. In still other embodiments, the biological sample may consist of saliva, cerebrospinal fluid, lymph, sweat, ucus, synovial fluid, lacrimal fluid or tears, urine, or other clinical specimens and samples, including bacterial or viral cultures.

The term “inductive effect,” as used herein, refers to the polarization of a chemical bond caused by the polarization of an adjacent bond. An inductive effect may be positive or negative. The term “mesomeric effect,” as used herein, refers to a resonance effect resulting from differences in electron density caused by electron delocalization in a chemical structure. A mesomeric effect may be positive or negative.

The term “specifically binds,” as used herein, means that two molecules form a complex that is relatively stable under physiologic conditions (e.g., a stable antigen/antibody complex). The term is also applicable where, for example, an antigen-binding domain is specific for a particular epitope, which is found on a number of molecules. Thus, an antibody may specifically bind multiple proteins when it binds to an epitope present in each. Specific binding is characterized by a selective interaction, often including high affinity binding with a low to moderate capacity. Nonspecific binding usually is a less selective interaction, and may have a low affinity with a moderate to high capacity. Typically, binding is considered specific when the affinity is at least 10⁵ M⁻¹, 10⁶ M⁻¹, 10⁷ M⁻¹ or 10⁸ M⁻¹. If necessary, non-specific binding can be reduced without substantially affecting specific binding by varying the binding conditions. Such conditions are known in the art, and a skilled artisan using routine techniques can select appropriate conditions. The conditions are usually defined in terms of concentration of antibodies, ionic strength of the solution, pH, temperature, time allowed for binding, concentration of non-related molecules (e.g., blocking agents such as serum albumin or milk casein), and so forth.

The term “subject,” as used herein, means an animal, including a human or non-human mammal, e.g., a dog, a cat, a mouse, a rat, a cow, a sheep, a pig, a goat, or a non-human primate, and expressly includes widely available laboratory mammals, livestock, and domestic mammals. In some embodiments, the mammal may be a human; in others, the mammal may be a rodent, such as a mouse or a rat. The term also includes, but is not limited to, other commonly used eukaryotic and prokaryotic experimental organisms or cell lines, such as zebrafish, Caenorhabditis elegans, Drosophila melanogaster, Saccharomyces cerevisiae, HeLa, Escherichia coli, and the like.

### Exemplary Embodiments

In one aspect, the present invention provides compositions for specifically modifying, isolating, detecting, and quantifying citrulline and/or homocitrulline-containing peptides, polypeptides, and proteins. The compositions comprise citrulline and/or homocitrulline-reactive mono- and disubstituted glyoxal derivatives of formula (I):

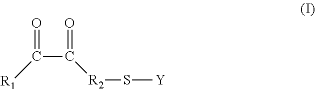

In certain embodiments, R₁ and R₂ comprise any branched or unbranched alkyl or aryl chains of different size, length, hydrophobicity, water-solubility, of different positive or negative inductive effect, of different positive or negative mesomeric effect, or just a hydrogen. For example, R₁ could be selected from the group consisting of —H, —CH₃, —CH₂CH₃, phenyl, and other derivatives thereof. Similarly, R₂ could be selected from the group consisting of —H, —CH₃, —CH₂CH₃, phenyl, and other derivatives thereof.

S in formula (I) represents a spacer. The spacer S exposes the citrulline and/or homocitrulline-reactive group to solution without changing its reactivity or specificity, and the molecular tag Y to solution without changing its binding affinity or specificity. The spacer S also affects the solubility of the citrulline and/or homocitrulline-reactive compounds. The spacer S comprises any branched or unbranched aliphatic or polyethylene glycol-based chain of variable size, length, and hydrophobicity. The spacer S may also contain a cleavage site, such as disulfide linkages (cleaved by dithiothreitol (DTT)), photocleavable groups (cleaved by light, such as o-nitrobenzyl, or pivaloyl), base labile groups, or enzymatic sites (e.g., peptide sequences cleaved by specific proteolytic enzymes, such as trypsin, chymotrypsin, Lys-C, Asp-N, Glu-C and the like). In certain embodiments, the spacer S is an alkyl spacer, such as —(CH₂)ₙ— or a PEG-derived spacer, such as —(O—CH₂CH₂)ₙ—, wherein n=1 to 25.

Y in formula (I) is a physical or molecular tag that facilitates identification, visualization, detection or purification of citrulline and/or homocitrulline-containing peptides, polypeptides or proteins labeled with the compound. In certain embodiments, Y is a magnetic bead, resin, or other solid support, and the citrulline and/or homocitrulline-reactive compounds can be used to isolate citrulline and/or homocitrulline-containing peptides, polypeptides, or proteins from complex mixtures, such as biological samples. In certain other embodiments, Y is selected from the group consisting of biotin, iminobiotin, biotinyl-6-aminohexanoic acid, a His-tag, a metal affinity tag (SEQ ID NO:1), a FLAG peptide (SEQ ID NO:2), digoxin, a dinitrophenyl group, a nitrotyrosine residue, fluorescein isothiocyanate, Texas Red, and rhodamine. In certain other embodiments, Y is a radioisotope tag, such as ¹²⁵I, a signature ion tag to produce a specific fragment ion in MS/MS experiments or in MALDI imaging, such as a biotinyl residue or a short peptide sequence highly prone to fragmentation upon MS/MS analysis. In certain embodiments, Y is a citrulline and/or homocitrulline-specific label transfer reagent for mapping protein-protein interactions, such as those commercially available from Pierce (Rockford, Ill., U.S.A.). The citrulline and/or homocitrulline-reactive compounds of such embodiments can be used to identify, visualize, purify or detect citrulline and/or homocitrulline-containing peptides, polypeptides, or proteins in complex mixtures, such as biological samples.

Representative mono- or disubstituted glyoxal derivatives of formula (I) include

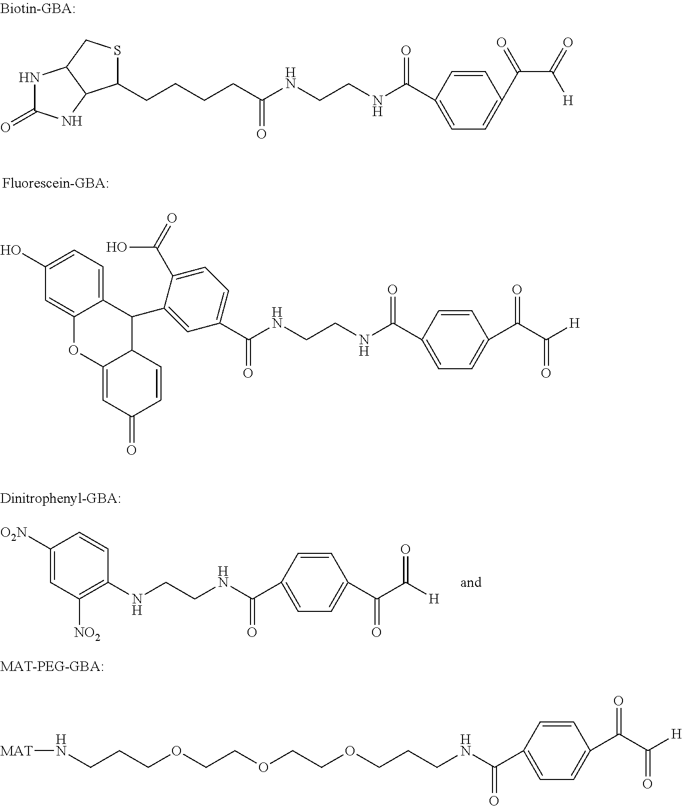

Such compounds are currently synthesized using conventional solid phase chemistry on Nova Tag™ resins purchased from Merck & Co., Inc. (Whitehouse Station, N.J., USA). The Nova Tag™ resins have the general structure:

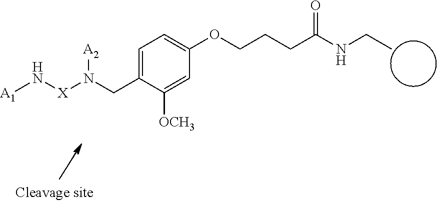

Biotin-PEG-GBA was synthesized using the Biotin-PEG Nova Tag™ resin, which was preloaded with biotin (A₁) and a PEG linker (X). The GBA (A₂) was coupled as described in paragraph 80 of Example 2. The resin was then washed and the Biotin-PEG-GBA was cleaved from the resin using trifluoroacetic acid (TFA). Other combinations of A₁, A₂, and X can be generated using the Universal Nova Tag™ or the Universal PEG Nova Tag™ (X=PEG) resins. Those resins are available with different protecting groups at the A₁ (Mmt, or monomethoxytrityl) and A₂ (Fmoc, or fluorenylmethoxycarbonyl) positions which are cleaved under different conditions, permitting the attachment of different groups sequentially. To synthesize different citrulline and/or homocitrulline-reactive mono- and disubstituted glyoxal derivatives by this method, the Fmoc protecting group is first cleaved with 20% piperidine and the desired A₂ is added using HATU (O-(7-Azabenzotriazole-1-yl)-N,N,N′N′-tetramethyluroniu hexafluorophosphate) or PyBOP (benzotriazol-1-yl-oxytripyrrolidino-phosphonium hexafluorophosphate) and DIPEA (N,N-diisopropylethylamine). Subsequently, the Mmt protecting group is cleaved using 0.6 M HOBt in DCM/TFE (1:1) for 1 hour, and the desired A₁ is added using PyBOP in the presence of DIPEA.

In another aspect, the present invention provides methods of modifying citrulline and/or homocitrulline-containing peptides, polypeptides, and proteins under acidic conditions. The methods comprise contacting a solution or sample, such as a biological sample, comprising at least one citrulline and/or homocitrulline-containing peptide, polypeptide, or protein, and at least one citrulline and/or homocitrulline-reactive compound of foimula (I), as described in paragraphs [051] to [053] above, wherein the citrulline and/or homocitrulline-reactive compound becomes covalently attached to at least one citrulline and/or homocitrulline residue within the at least one citrulline and/or homocitrulline-containing peptide, polypeptide, or protein in the solution or sample. In one embodiment, the peptide, polypeptide or protein comprises citrulline. In another embodiment, the peptide, polypeptide or protein comprises homocitrulline.

In certain embodiments, the solution or sample and the at least one citrulline and/or homocitrulline-reactive compound are incubated, for example, at a temperature between 4° C. and 70° C. for a sufficient period of time for the citrulline and/or homocitrulline-modification reaction to occur. In certain embodiments, the solution or sample and the at least one citrulline and/or homocitrulline-reactive compound can be incubated at 4° C., 10° C., 15° C., 20° C., 25° C., 37° C., 45° C., 47° C., 55° C., 60° C., 65° C., or 70° C. In certain other embodiments, the solution or sample and the at least one citrulline and/or homocitrulline-reactive compound can be incubated at the desired temperature for 1, 2, 3, 4, 5, 6, 7, 8, 9, 10, 12, 15, 20, or 24 hours, or for longer times as necessary.

In certain embodiments, the sample comprising at least one citrulline and/or homocitrulline-containing peptide, polypeptide, or protein is a biological sample. In certain embodiments, the biological sample is a tissue biopsy taken, for example, from the cerebrospinal fluid of a patient afflicted with multiple sclerosis, or from the synovium of a patient afflicted with rheumatoid arthritis. In one embodiment, the biological sample is synovial fluid. In certain other embodiments, the biological sample is selected from cultured cells, bacterial or viral cultures, cerebrospinal fluid, serum, blood, plasma, saliva, amniotic fluid, lacrimal fluid or tears, milk, lymph, urine, and sweat. Other permutations and possibilities for selecting other appropriate types of biological samples will be readily apparent to one of ordinary skill in the art.

In another aspect, the present invention provides methods for isolating, enriching, or purifying citrulline and/or homocitrulline-containing peptides, polypeptides, or proteins from a solution or sample. The methods comprise contacting at least one citrulline and/or homocitrulline-reactive compound of formula (I), as described in paragraphs [051] to above, with a solution or sample containing at least one citrulline and/or homocitrulline-containing peptide, polypeptide, or protein; wherein the citrulline and/or homocitrulline-reactive compound becomes covalently attached to at least one citrulline and/or homocitrulline residue within the at least one citrulline and/or homocitrulline-containing peptide, polypeptide, or protein in the solution or sample; collecting the modified citrulline and/or homocitrulline-containing peptides, polypeptides, or proteins; and removing the unmodified peptides, polypeptides, or proteins from the solution or sample. In one embodiment, the peptide, polypeptide or protein comprises citrulline. In another embodiment, the peptide, polypeptide or protein comprises homocitrulline.

In certain embodiments, the sample comprising at least one citrulline and/or homocitrulline-containing peptide, polypeptide, or protein is a biological sample. In certain embodiments, the biological sample is a tissue biopsy taken, for example, from the cerebrospinal fluid of a patient afflicted with multiple sclerosis, or from the synovium of a patient afflicted with rheumatoid arthritis. In one embodiment, the biological sample is synovial fluid. In certain other embodiments, the biological sample is selected from cultured cells, bacterial or viral cultures, cerebrospinal fluid, serum, blood, plasma, saliva, amniotic fluid, lacrimal fluid or tears, milk, lymph, urine, and sweat. Other permutations and possibilities for selecting other appropriate types of biological samples will be readily apparent to one of ordinary skill in the art.

In certain embodiments, the citrulline and/or homocitrulline-reactive compounds of formula (I), as described in paragraphs [051] to [053] above, are used to facilitate purification of modified citrulline and/or homocitrulline-containing peptides, polypeptides, or proteins from complex mixtures, such as biological samples. In certain embodiments, the citrulline and/or homocitrulline-reactive compounds of formula (I) comprise a physical or molecular tag Y, wherein Y is a compound that specifically binds avidin/streptavidin, monomeric avidin, NeutrAvidin™ (available from Pierce, Rockford, Ill., USA), and the like, such as biotin or iminobiotin. The modified citrulline and/or homocitrulline-containing peptides, polypeptides, or proteins can then be isolated using magnetic or other beads coated with avidin/streptavidin, as described in Example 2. Alternatively, any of a number of commonly used chromatography substrates can be similarly coated with avidin/streptavidin and used to isolate the modified citrulline and/or homocitrulline-containing peptides, polypeptides, or proteins. In one embodiment, the peptide, polypeptide or protein comprises citrulline. In another embodiment, the peptide, polypeptide or protein comprises homocitrulline.

In certain other embodiments, the citrulline and/or homocitrulline-reactive compounds of formula (I) comprise a physical or molecular tag Y, wherein Y is a peptide tag, such as polyhistidine (“His tag”) (e.g., (His)₆₋₈) or a metal affinity tag (“MAT”) (SEQ ID NO:1). The modified citrulline and/or homocitrulline-containing peptides, polypeptides, or proteins can then be isolated by metal affinity chromatography, for example, using a Ni-NTA column. In certain other embodiments, the citrulline and/or homocitrulline-reactive compounds of formula (I) comprise a physical or molecular tag Y, wherein Y is a compound capable of being bound by various commercially available antibodies or antibody fragments, e.g., biotin, the FLAG peptide (SEQ ID NO:2), digoxin, a dinitrophenyl group, or a nitrotyrosine residue. The modified citrulline and/or homocitrulline-containing peptides, polypeptides, or proteins can then be isolated by immunoprecipitation, or by immunoaffinity chromatography using beads or chromatography substrates derivatized with the appropriate antibody or antibody fragment. These methods can be used alone, or in conjunction with other routine methods of protein purification known to one skilled in the art, such as size fractionation, density gradient centrifugation, ultrafiltration, ammonium sulfate precipitation, cation or anion exchange chromatography, and the like, if necessary to reduce the complexity of the solution or sample comprising at least one citrulline and/or homocitrulline-containing peptide, polypeptide, or protein.

In certain other embodiments, a citrulline and/or homocitrulline-reactive compound with an appropriate functional group in addition to the mono- or disubstituted glyoxal derivative, such as 4-glyoxalbenzoic acid, can be covalently attached to an appropriately derivatized chromatography substrate or magnetic bead comprising a cleavable spacer, such as TentaGel S HMB resin, as described in Example 3. A solution or sample comprising at least one citrulline and/or homocitrulline-containing peptide, polypeptide, or protein can then be incubated with the modified magnetic beads or passed over a column containing the modified chromatography substrate under acidic conditions, thereby allowing the citrulline and/or homocitrulline-reactive group on the resin or beads to react with and modify the at least one citrulline and/or homocitrulline-containing peptide, polypeptide, or protein in the solution or sample. Next, the column or beads can be washed with a suitable buffer to remove any non-citrulline and/or homocitrulline-containing peptides, polypeptides, or proteins from the solution. The modified and immobilized peptides, polypeptides, or proteins can then be eluted or cleaved from the column or beads and collected for analysis by MS, or any other suitable method known to one skilled in the art. In one embodiment, the peptide, polypeptide or protein comprises citrulline. In another embodiment, the peptide, polypeptide or protein comprises homocitrulline.

In certain embodiments, the sample is a biological sample. In certain other embodiments, the biological sample is selected from a tissue biopsy, cultured cells, bacterial or viral cultures, cerebrospinal fluid, serum, blood, plasma, saliva, amniotic fluid, synovial fluid, lacrimal fluid or tears, milk, lymph, urine, and sweat. In one embodiment, the biological sample is synovial fluid. In certain embodiments, the citrulline and/or homocitrulline-reactive group is 4-glyoxalbenzoic acid. In certain embodiments, the resin comprising a cleavable spacer is TentaGel S 4-hydroxymethylbenzoic acid resin. In certain embodiments, the magnetic beads comprising a cleavable spacer are M-280 4-hydroxymethylbenzoic acid Dynabeads®.

Alternatively, samples comprising citrulline and/or homocitrulline-containing peptides, polypeptides, or proteins may be adsorbed to standard polystyrene plates used for enzyme-linked immunosorbent assays (ELISAs). The adsorbed samples could then be treated with a biotinylated citrulline and/or homocitrulline-reactive compound and detected with streptavidin conjugated to alkaline phosphatase or horseradish peroxidase. The presence or absence of citrulline and/or homocitrulline-containing peptides, polypeptides, or proteins would be detected by standard methods, for example, using a spectrophotometer and monitoring absorbance at 450 nm. In one embodiment, the peptide, polypeptide or protein comprises citrulline. In another embodiment, the peptide, polypeptide or protein comprises homocitrulline.

In another aspect, the present invention provides methods of detecting or visualizing citrulline and/or homocitrulline-containing peptides, polypeptides, or proteins in a biological sample. Modified citrulline and/or homocitrulline-containing peptides, polypeptides, or proteins can be directly detected, for example, on a gel or blot (e.g., a Western blot), or in tissue samples or sections (e.g., by in situ immunohistochemistry). In certain embodiments, the biological sample is a tissue biopsy taken, for example, from the cerebrospinal fluid of a patient afflicted with multiple sclerosis, or from the synovium of a patient afflicted with rheumatoid arthritis. In one embodiment, the biological sample is synovial fluid. In certain other embodiments, the biological sample is selected from serum, blood, plasma, saliva, amniotic fluid, synovial fluid, lacrimal fluid, milk, lymph, urine, and sweat. Other permutations and possibilities for selecting other appropriate types of biological samples will be readily apparent to one of ordinary skill in the art.

In certain embodiments, the citrulline and/or homocitrulline-reactive compounds of formula (I), as described in paragraphs [051] to [053] above, are used to identify or detect modified citrulline and/or homocitrulline-containing peptides, polypeptides, or proteins in solutions or samples. In one embodiment, the peptide, polypeptide or protein comprises citrulline. In another embodiment, the peptide, polypeptide or protein comprises homocitrulline. In certain embodiments, the citrulline and/or homocitrulline-reactive compounds of formula (I) comprise a physical or molecular tag Y, wherein Y is a compound that facilitates detection of the modified citrulline and/or homocitrulline-containing peptides, polypeptides, or proteins, for example, a tag recognized by commercially available antibodies, such as biotin, the FLAG peptide (SEQ ID NO:2), digoxin, a dinitrophenyl group, or a nitrotyrosine residue, and the like.

The modified citrulline and/or homocitrulline-containing peptides, polypeptides, or proteins can then be identified or visualized using commercially available antibodies modified with appropriate conjugates to identify cognate ligands by colorimetric, autoradiographic, or other types of detection. For example, the modified citrulline and/or homocit ulline-containing peptides, polypeptides, or proteins can be detected colorimetrically using antibodies conjugated with horseradish peroxidase and tetramethylbenzidine, or using alkaline phosphatase and any suitable phosphatase substrate. If detected colorimetrically, the amount of color may be measured using a luminometer, a spectrophotometer, or other similar instruments. If detected autoradiographically, the amount of signal may be measured from exposed x-ray film using a densitometer, directly from a gel or blot using a PhosphorImager® or similar instrument, or in solution using a luminometer. These methods can be used alone, or in conjunction with other routine methods of detecting proteins known to one skilled in the art, such as Western blotting or in situ immunohistochemistry. Such methods may further comprise fixation of proteins, either in situ (e.g., by formalin fixation, paraffin-embedding, and thin sectioning), or by electrophoresis and blotting (e.g., by SDS-PAGE and Western blotting) Other permutations and possibilities for selecting additional appropriate methods of detection will be readily apparent to one of ordinary skill in the art.

In certain other embodiments, the citrulline and/or homocitrulline-reactive compounds of formula (I) comprise a physical or molecular tag Y, wherein Y is a fluorophore or chromophore, such as fluorescein isothiocyanate, Texas Red, rhodamine, or the like. The modified citrulline and/or homocitrulline-containing peptide, polypeptide, or protein can then be identified or visualized by routine methods known to one skilled in the art, such as fluorescence microscopy or spectroscopy, with a luminometer or spectrophotometer, by liquid chromatography with fluorescence detection, and the like. These methods can be used alone, or in conjunction with other routine methods of detecting proteins known to one skilled in the art, such as Western blotting or in situ immunohistochemistry. Such methods may further comprise fixation of proteins, either in situ (e.g., by formalin fixation, paraffin-embedding, and thin sectioning), or by electrophoresis and blotting (e.g., by SDS-PAGE and Western blotting). Other permutations and possibilities for selecting additional appropriate methods of detection will be readily apparent to one of ordinary skill in the art.

In another aspect, the present invention provides methods of quantifying the relative amounts of citrulline and/or homocitrulline-containing peptides, polypeptides, or proteins in different biological samples, taken, for example, from healthy and diseased individuals, or taken from a diseased individual before and after receiving a particular treatment. In one embodiment, the peptide, polypeptide or protein comprises citrulline. In another embodiment, the peptide, polypeptide or protein comprises homocitrulline.

This method uses a citrulline and/or homocitrulline-reactive compound synthesized in both “light” and “heavy” versions, where Y might be biotin. For example, Biotin-PEG-GBA can be synthesized with ¹²C or ¹³C at all six carbons in the phenyl ring of GBA. Thus, in a “light” version of Biotin-PEG-GBA, all six phenyl carbons would be ¹²C. In a “heavy” version of Biotin-PEG-GBA, all six phenyl carbons would be ¹³C. The “light” and “heavy” versions of Biotin-PEG-GBA are distinguishable by mass spectrometry, such that the “heavy” compound (or a citrulline and/or homocitrulline-containing peptide, polypeptide, or protein modified with the “heavy” compound), is shifted by +6 Da from the “light” compound (or a citrulline and/or homocitrulline-containing peptide, polypeptide, or protein modified with the “light” compound) for each modified citrulline and/or homocitrulline. One skilled in the art would appreciate that other isotope pairs, such as ¹H/²H, may also be used to synthesize “light” and “heavy” versions of citrulline and/or homocitrulline-reactive compounds for use with the methods of the invention.

In certain embodiments, the citrulline and/or homocitrulline-containing peptides, polypeptides, or proteins in a first biological sample will be modified with the light version of a citrulline and/or homocitrulline-reactive compound, such as Biotin-PEG-GBA, and the citrulline and/or homocitrulline-containing peptides, polypeptides, or proteins in a second biological sample will be modified with the heavy version of the same compound used with the first biological sample. The first and second biological samples are then mixed together and can then be digested with trypsin or other appropriate reagent. The modified and biotinylated citrulline and/or homocitrulline-containing peptides, polypeptides, or proteins can be collected from the mixed first and second biological samples, for example, using streptavidin-coated M-280 Dynabeads® or M-270 Dynabeads®. The modified peptides isolated from the mixed first and second biological samples can then be analyzed by MALDI-TOF/TOF or LC-tandem mass spectrometry, thereby determining the relative amount of each citrulline and/or homocitrulline-containing peptide, polypeptide, or protein in the first and second biological samples. In parallel, the identity of each modified peptide, polypeptide, or protein can be determined by MS/MS.

In certain embodiments, the biological sample is selected from a tissue biopsy, cultured cells, bacterial or viral cultures, cerebrospinal fluid, serum, blood, plasma, saliva, amniotic fluid, synovial fluid, lacrimal fluid or tears, milk, lymph, urine, and sweat. In one embodiment, the biological sample is synovial fluid.

In certain embodiments, this method can be used to evaluate the efficacy of a treatment for a disease associated with altered citrullination of at least one citrulline and/or homocitrulline-containing peptide, polypeptide, or protein. In one embodiment, the peptide, polypeptide or protein comprises citrulline. In another embodiment, the peptide, polypeptide or protein comprises homocitrulline. A biological sample can be taken from a patient suffering from such a disease before and after administration of a treatment. The relative amounts of at least one citrulline and/or homocitrulline-containing peptide, polypeptide, or protein can be determined by the method described above, and the efficacy of the treatment can be assessed. In certain embodiments, the biological sample is selected from a tissue biopsy, cultured cells, bacterial or viral cultures, cerebrospinal fluid, serum, blood, plasma, saliva, amniotic fluid, synovial fluid, lacrimal fluid or tears, milk, lymph, urine, and sweat. In one embodiment, the biological sample is synovial fluid. In certain embodiments, the disease associated with altered citrullination of at least one citrulline and/or homocitrulline-containing peptide, polypeptide, or protein is multiple sclerosis. In certain other embodiments, the disease associated with altered citrullination of at least one citrulline and/or homocitrulline-containing peptide, polypeptide, or protein is rheumatoid arthritis.

The methods of the invention may also be implemented in kits incorporating one or more of the above techniques to isolate, detect, or quantify citrulline and/or homocitrulline-containing peptides, polypeptides, or proteins from a solution or biological sample. For example, in one embodiment, a kit provides light and heavy versions of a citrulline and/or homocitrulline-reactive compound, such as Biotin-PEG-GBA. The kit further provides all necessary tubes, containers or reaction vessels, buffers and reagents required to perform the various steps of the method, including avidin/streptavidin coated beads for isolating the modified, biotinylated citrulline and/or homocitrulline-containing peptides, polypeptides, or proteins. The beads and other materials can be provided in an appropriate buffer or solvent, or as lyophilized powders. Similarly, the buffers and other reagents may be provided premixed, or in dry form that must be reconstituted by the user of the kit. Such kits can also contain other components, including packaging, instructions, or other material to aid in the implementation of the method.

In another embodiment, kits provide mono- or disubstituted glyoxal derivatives suitable for the isolation, enrichment, or purification of citrulline and/or homocitrulline-containing peptides, polypeptides, or proteins from a complex mixture, such as a solution or biological sample. For example, in one embodiment, a kit provides magnetic or other beads, resins, or other solid supports derivatized with a citrulline and/or homocitrulline-reactive compound. The kit further provides all necessary tubes, containers or reaction vessels, buffers and reagents required to perform various steps of the method, including chromatography columns or the like, in order to isolate, enrich, or purify the desired citrulline and/or homocitrulline-containing peptides, polypeptides, or proteins.

In still another embodiment, a kit provides mono- or disubstituted glyoxal compositions suitable for the detection or visualization of citrulline and/or homocitrulline-containing peptides, polypeptides, or proteins from a complex mixture, such as a solution or biological sample. For example, in one embodiment, the kit contains Biotin-PEG-GBA, and the citrulline and/or homocitrulline-containing peptides, polypeptides, or proteins are detected or visualized using streptavidin conjugated with alkaline phosphatase or horseradish peroxidase by standard methods. In certain embodiments, the biological sample undergoes an additional fractionation step, such as chromatography, filtration, precipitation (e.g., by salt, pH, or organic solvent), and gel electrophoresis and Western blotting, or formalin fixation, paraffin-embedding, and sectioning. In such embodiments, the kit further provides all necessary tubes, containers or reaction vessels, buffers and reagents required to detect or visualize citrulline and/or homocitrulline-containing peptides, polypeptides, or proteins, whether on a solid filter (e.g., Western blotting), in a tissue section, or the like.

### EXAMPLES

### Reagents and Materials

2,3-butanedione (purity >99%), Boc-Cit-OH (technical grade, >84%), and Silica Gel 60 were purchased from Fluka Production GmBH (Buchs, Switzerland). Trifluoroacetic acid (TFA) and DMF were purchased from Fluka (St. Louis, Mo., USA). Hen egg lysozyme, ovalbumin, glacial acetic acid, α-cyano-4-hydroxycinnamic acid (“α-CHCA,” purity >99%), 4-acetylbenzoic acid, methylglyoxal, phenylglyoxal, potassium cyanate, sodium dodecyl sulfate (SDS), and selenium dioxide were purchased from Sigma-Aldrich (St. Louis, Mo., USA). Biotin NovaTag resin was purchased from Novabiochem (Merck, Darmstadt, Gei many). Streptavidin-HRP was obtained from Southern Biotech (Birmingham, Ala., USA). Water was supplied by a Milli RX 45 water purification system from Millipore (Billerica, Mass., USA). Instrument grade nitrogen (5.0), and argon (5.0) were purchased from AGA (Oslo, Norway). The polypeptides SAVRA-Cit-SSVPGVR and SAVRL-R-SSVPGVR were purchased from Genscript (Piscataway, N.J., USA).

MALDI TOF/TOF and nanoLC-qTOF MS/MS Procedures

Samples were analyzed using an Ultraflex II MALDI TOF/TOF instrument (Bruker Daltonics, Bremen, Germany) operated in the positive reflectron mode, using a solution of 5 mg/mL α-CHCA in acetonitrile/0.1% TFA (70/30, v/v) as matrix. Alternatively, samples were analyzed using a microTOF-Q MS/MS instrument (Bruker Daltonics) equipped with interfaces for both off-line nanospray and nanoscale liquid chromatography supplied by Agilent Technologies (Santa Clara, Calif., USA).

### Example 1

### Modification of Citrulline-Containing Peptides, Polypeptides, and Proteins with Mono- and Disubstituted Glyoxal Derivatives

### Modification of Citrulline Using 2,3-Butanedione and Biotin Derivatives

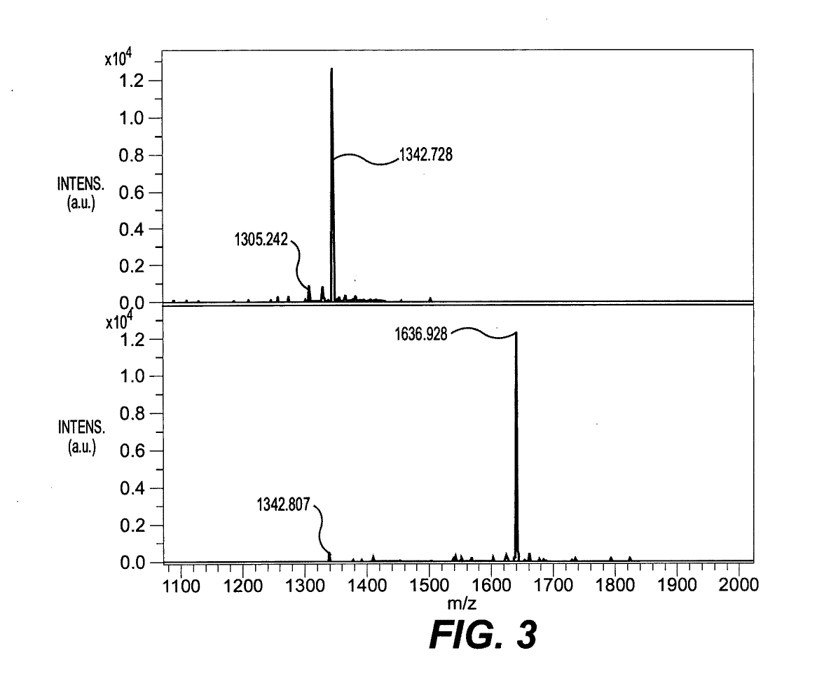

Solutions of 100 μM polypeptide (e.g., SAVRA-Cit-SSVPGVR) and 50 mM 2,3-butanedione were prepared in water; 50 mM solutions of various biotin derivatives, including Sulfo-NHS-LC-Biotin (Invitrogen, Carlsbad, Calif., USA), Sulfo-NHS-SS-Biotin and Sulfo-NHS-Biotin (both from Pierce, Rockford, Ill., USA), were prepared in 100% TFA. Solutions containing 2,3-butanedione were freshly prepared in dark-colored tubes prior to modification. The polypeptide modification reaction contained: 10 μL 100 μM polypeptide, 20 μL 100% TFA, 10 μL 50 mM biotin derivative and 10 μL 2,3-butanedione. The reaction was mixed in a dark-colored microcentrifuge tube and incubated at 37° C. for 3 hours. Upon completing the incubation, the reaction mixture was dried under vacuum in a Speed-Vac. The pellet was dissolved in 50 μL of 0.1% TFA and analyzed by Matrix-Assisted Laser Desorption/Ionization Time-of-Flight Mass Spectrometry (“MALDI-TOF MS”). The resulting spectrum is shown in FIG. 3. The top spectrum displays a singly-charged peak at m/z 1342.7, which corresponds to unmodified SAVRA-Cit-SSVPGVR polypeptide. The bottom spectrum displays a dominating, singly-charged peak at m/z 1636.9, corresponding to a mass shift of +294.1 Da. The reactive intermediate foamed by modification of a polypeptide containing a single citrulline residue using 2,3-butanedione alone produces a mass shift of +50 Da. Consequently, a mass shift of +294.1 Da corresponds to the formation of the reactive intermediate (+50 Da) with D-biotin (M.W.=244.1 g/mole).

The polypeptide SAVRA-Cit-SSVPGVR was also modified by the reaction conditions described above, with 2,3-butanedione, but replacing D-biotin with biotinyl-aminohexanoic acid. Biotinyl-aminohexanoic acid was used to provide a linker or spacer between the polypeptide and the biotin group, in an effort to optimize avidin/streptavidin binding. The MALDI-TOF MS spectrum again shows a single dominating peak at m/z 1749.96, corresponding to a mass shift of +407.1 Da (data not shown). That mass shift corresponds to generation of the reactive intermediate (+50 Da) with biotinyl-aminohexanoic acid (M.W.=357.1 g/mole).

### Depletion of Modified SAVRA-Cit-SSVPGVR by Streptavidin

A purification method using strong cation exchange (“SCX”) chromatography and reversed phase (“RP”) solid phase extraction was developed to remove excess biotin or biotin derivatives. According to this method, the reaction mixture was dried under vacuum in a Speed-Vac to remove the TFA, and resuspended in a mixture of equal parts 100% acetonitrile and 10 mM foimic acid to enable the modified polypeptides to bind a microcolumn containing polysulfoaspartamide (“PSA”) (PolyLC, Inc.), the SCX material. The positively-charged polypeptides bind the resin, while the negatively-charged biotin and biotin derivatives do not, and thus can be washed away. The PSA column was washed with 100 μL of the 100% acetonitrile/10 mM formic acid mixture. The purified polypeptides were then eluted from the PSA column using a mixture of 30% acetonitrile/70% 2M NaCl. The acetonitrile was subsequently removed by evaporation to facilitate polypeptide binding in the subsequent RP purification, using a Poros® R2 reversed phase chromatography column, with a 20 μm particle size and a 2000 Å pore size (Applied Biosystems, Foster City, Calif.). The peptides were eluted from the RP column with 20 μL of 70% acetonitrile/30% 10 mM formic acid.

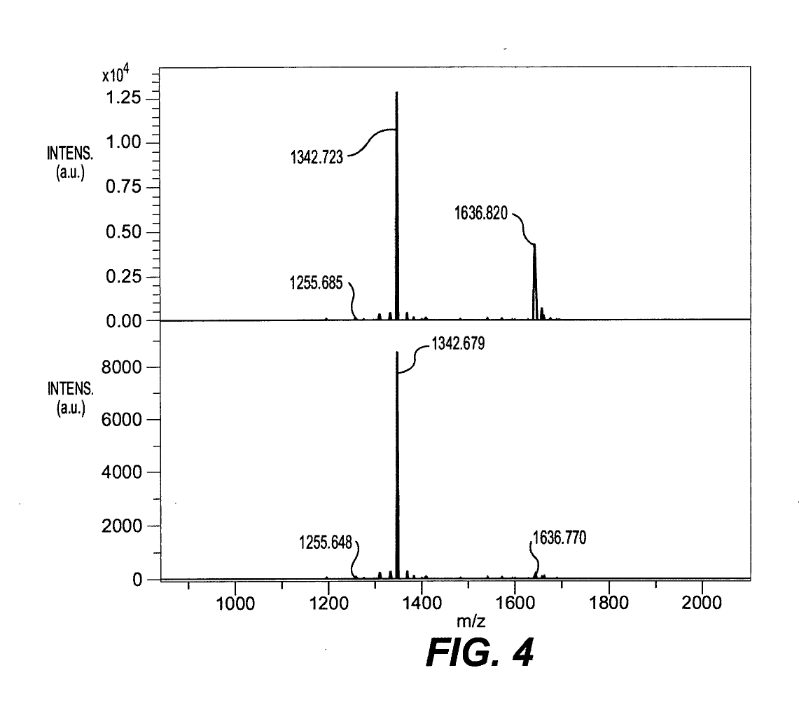

The resulting fraction was again dried under vacuum in a Speed-Vac and dissolved in water to a final concentration of 100 μM modified polypeptide. Subsequently, 5 μL 100 μM biotinylated SAVRA-Cit-SSVPGVR polypeptide was mixed with 5 μL 100 μM unmodified SAVRA-Cit-SSVPGVR polypeptide and added to 90 μL of 1× phosphate-buffered saline (“PBS”) (1×PBS contains 137 mM NaCl, 2.7 mM KCl, 10 mM Na₂HPO₄, and 2 mM KH₂PO₄, at pH=7.4). 20 μL of the polypeptide mixture was added to 50 μL of 2 mg/mL streptavidin coated magnetic M-280 Dynabeads® (Invitrogen, Carlsbad, Calif.) after first washing the beads five times with 50 μL 1×PBS. The polypeptide-bead mixture was incubated at room temperature (“RT”) for 30 minutes with gentle resuspension of the beads every 5 minutes. After thirty minutes, the beads were magnetically collected and an aliquot of the supernatant was measured by MALDI-TOF MS. The resulting spectrum is shown in FIG. 4. The upper spectrum displays two peaks: one at m/z 1342.7 corresponding to the unmodified SAVRA-Cit-SSVPGVR polypeptide, and one at m/z 1636.8 corresponding to the biotinylated SAVRA-Cit-SSVPGVR polypeptide. The lower spectrum displays a single peak at m/z 1342.7, showing that the biotinylated polypeptide bound quantitatively to the streptavidin-labeled beads. Similar results were obtained when this experiment was repeated with polypeptides modified with biotinyl-aminohexanoic acid (data not shown). Elution of biotinylated molecules from streptavidin beads can be difficult, however, because of the extremely high affinity of the biotin/streptavidin interaction. Therefore commercially available alternatives that enable elution of the bound polypeptides may also be used. Alternatively, biotin derivatives containing a cleavable linker may also be used.

### Specificity of the Modification for Citrulline

To investigate the specificity of the reaction described above, peptides covering all twenty naturally occurring amino acids were subjected to identical reaction conditions. The peptides DGLAHLDNLKGTFATLSELH, EWMGGIIPIFGTANYAQKFRGR and CPKEIPKGSKNTEVL were used. After modification, the peptides were analyzed by MALDI TOF MS (data not shown). No modification was observed on any of the amino acids except for cysteine, as indicated by a mass shift of +68 Da in the peptide CPKEIPKGSKNTEVL (also assigned by MALDI TOF/TOF analysis) (data not shown). This mass shift is likely due to partial modification of the cysteine residue by 2,3-butanedione alone, resulting in a modified peptide lacking the biotin moiety, which therefore could not be enriched by avidin/streptavidin. Nevertheless, the variable modification of +68 Da on cysteines should be taken into consideration when employing search algorithms. In addition, arginine is commonly modified by 2,3-butanedione, albeit at slightly basic pH. The methods described here employ very acidic conditions under which no modification was observed on peptides containing arginine. Consequently, the experiments have shown that the biotinylation reaction is specific for citrulline residues.

### Modification of Citrulline Using Commercial Preparations of Methyl- and Phenylglyoxal

These experiments were performed using two pairs of polypeptides: (1) SAVRA-R-SSVPGVR and SAVRA-Cit-SSVPGVR; and (2) TGSSTGG-R-QGSHHE and TGSSTGG-Cit-QGSHHE. Each pair differed at a single position, with one polypeptide containing an arginine residue at that position, the other containing a citrulline residue. Solutions containing 100 μM SAVRA-R-SSVPGVR or SAVRA-Cit-SSVPGVR and 10 mM methylglyoxal were prepared in water. A series of reaction mixtures containing 10 μL of 100 μM polypeptide solution, 20 μl of TFA, and 10 μL of 10 mM methylglyoxal were prepared in 1.5 mL microcentrifuge tubes, producing working concentrations of 25 μM polypeptide, 50% TFA, and 2.5 mM methylglyoxal. The reaction solutions were incubated at 37° C., 45° C., 47° C., or 55° C. and analyzed by MALDI TOF MS after incubation for 1, 2, 4, or 6 hours, or overnight.

Similarly, solutions containing 100 μM of SAVRA-R-SSVPGVR or SAVRA-Cit-SSVPGVR polypeptide and 0.5 mM, 5 mM, 10 mM, or 50 mM phenylglyoxal were prepared in water. Reaction mixtures containing 10 μL of 100 μM polypeptide solution, 20 μL TFA, and 10 μL of 0.5 mM, 5 mM, or 10 mM phenylglyoxal were prepared in 1.5 mL microcentrifuge tubes, producing working concentrations of 25 μM polypeptide, 50% TFA, and 0.125 mM, 1.25 mM, and 2.5 mM phenylglyoxal, respectively. In addition, a reaction mixture containing 10 μL of 100 μM polypeptide solution, 20 μL, of TFA, 8 μL of 50 mM phenylglyoxal, and 2 μL water was prepared in a 1.5 ml microcentrifuge tube, producing working concentrations of 25 μM polypeptide, 50% TFA, and 10 mM phenylglyoxal. These reactions were incubated at 37° C. and 45° C. for 1, 1.5, 2, 2.5, 3, 4, or 5 hours, and then analyzed by MS (data not shown).

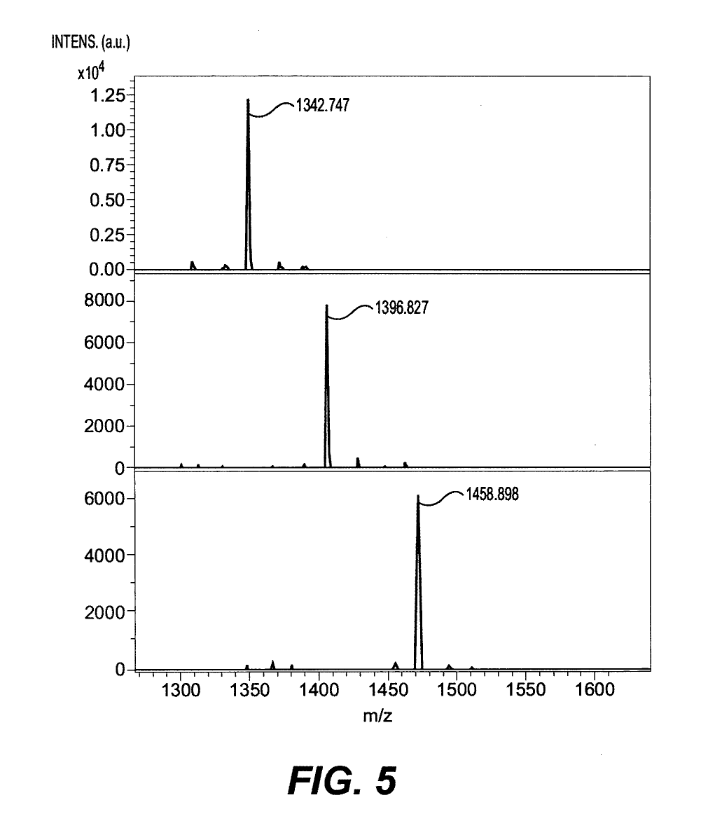

Nearly quantitative labeling was obtained using 2.5 mM methylglyoxal, or 10 mM phenylglyoxal, with optimal results in both cases requiring incubation for 4 to 5 hours at 45° C. These results for modification of the polypeptide SAVRA-Cit-SSVPGVR are shown in FIG. 5. The spectra in FIG. 5 were obtained from unmodified SAVRA-Cit-SSVPGVR (top), from SAVRA-Cit-SSVPGVR modified with methylglyoxal (middle), showing the expected mass shift of +54 Da, and from SAVRA-Cit-SSVPGVR modified with phenylglyoxal (bottom), showing the expected mass shift of +116 Da. These data show that both methyl- and phenylglyoxal derivatives can be used to specifically and quantitatively modify polypeptide-bound citrulline residues. Similar results were obtained with the TGSSTGG-R-QGSHHE and TGSSTGG-Cit-QGSHHE polypeptide pair (data not shown). Amino acid residues other than citrulline were not found to be modified under the reaction conditions described above.

The stability of the methylglyoxal modification was tested under basic conditions using 0.3 M NaOH. Methylglyoxal-modified SAVRA-Cit-SSVPGVR was dried under vacuum in a Speed-Vac to remove the TFA. A 5 μM solution of the modified polypeptide in water was prepared. A mixture containing 9 μL of the 5 μM polypeptide and 1 μL of 3 M NaOH was prepared in a microcentrifuge tube and incubated at room temperature for 1 or 2 hours. Those samples were then analyzed by MS. The methylglyoxal-modified SAVRA-Cit-SSVPGVR peptide was found to be stable during treatment with 0.3 M NaOH, showing that the chemical structure of citrulline modified by mono- or disubstituted glyoxal derivatives is stable under basic conditions.

### Synthesis of 4-Glyoxalbenzoic Acid (GBA)

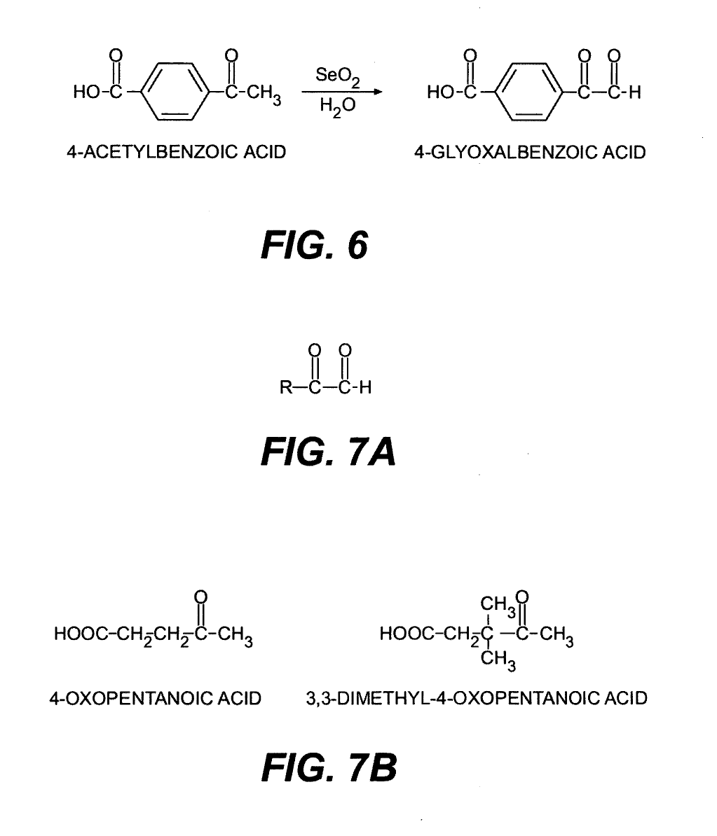

Next, 4-glyoxalbenzoic acid (GBA), a phenylglyoxal derivative including an additional carboxyl group was synthesized. GBA was prepared according to the scheme presented in FIG. 6. First, 20 mg (0.5 M) 4-acetylbenzoic acid and 14 mg (0.5 M) selenium dioxide (both from Sigma-Aldrich, St. Louis, Mo.) were suspended in 250 μl dioxane containing 2% (v/v) water. The sample mixture was incubated for 6 hours at 90° C. and cooled overnight at 4° C. The solid selenium precipitated out of solution, leaving a supernatant containing the product and any remaining reactant. The supernatant was loaded onto a silica gel column for purification of the product (the silica gel was purchased from Fluka, Buchs, Switzerland). Two different solvent systems were used to purify the GBA: A) acetone and ethylacetate (1:1) or B) acetone and ethylacetate (1:1) containing 1% acetic acid. System A was used to quantitatively elute the remaining reactant from the column. Next, system B was used to elute the oxidized product. Fractions of 1 ml were collected and analyzed by thin layer chromatography on 0.2 mm thick aluminum sheets coated with Silica Gel 60 F254 (Merck, Darmstadt, Germany), to test for the presence of GBA and to determine the purity of the product. This purification scheme has not yet been optimized, and the preliminary yield of GBA was 5 mg product.

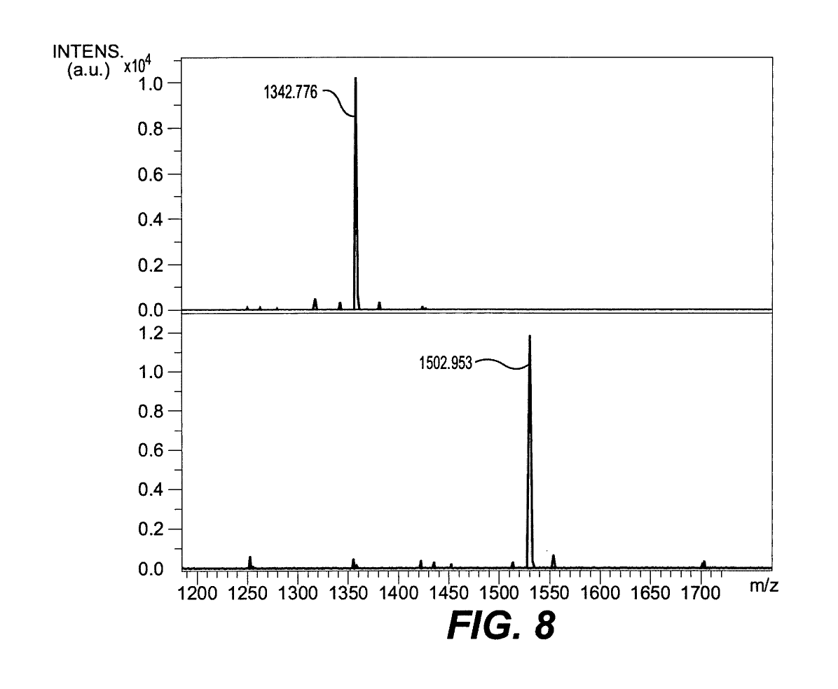

The general formula of monosubstituted glyoxal derivatives is shown in FIG. 7a. Several other commercial compounds are available which could serve as starting reagents for the synthesis of monosubstituted glyoxal derivates by oxidation with selenium dioxide, such as 4-oxopentanoic acid and 3,3-dimethyl-4-oxopentanoic acid, shown in FIG. 7b. The glyoxal derivatives corresponding to those compounds may be synthesized in the future to compare their reactivity and specificity for modification of citrulline-containing polypeptides.

### Modification of Citrulline Using Synthetic GBA

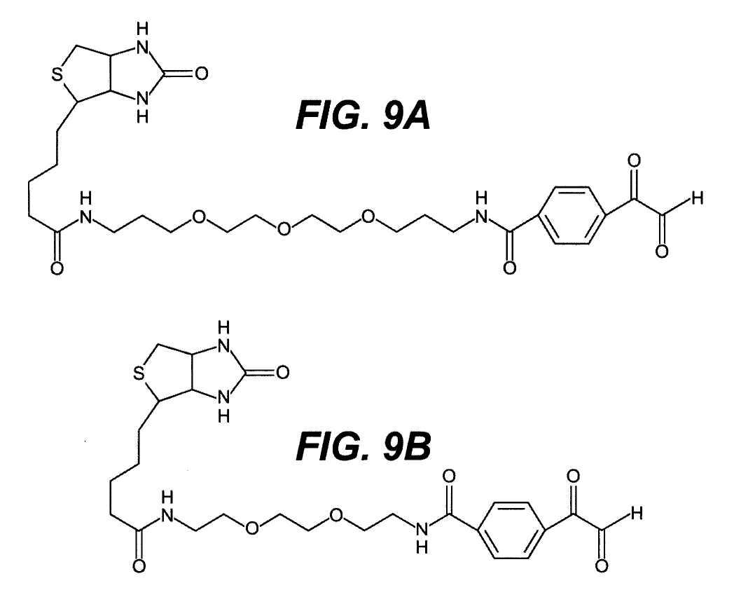

The purified GBA was used to modify the polypeptide SAVRA-Cit-SSVPGVR. The polypeptide SAVRA-R-SSVPGVR was used as a control. A reaction mixture containing 25 μM polypeptide and 10 mM GBA was incubated for 2 hours at 45° C. in 50% TFA. The reaction mixture was then purified by RP solid phase extraction. The polypeptides were analyzed by MALDI-TOF MS before and after modification. The results of the MS analysis are shown in FIG. 8. The spectrum of unmodified SAVRA-Cit-SSVPGVR appears as expected at m/z 1342.8 (FIG. 8, top). The spectrum for GBA-modified SAVRA-Cit-SSVPGVR shows a homogeneous product at m/z 1502.9, corresponding to the expected mass shift of +160 Da compared to the unmodified peptide (FIG. 8, bottom). The absence of a signal corresponding to the reactant (i.e., unmodified SAVRA-Cit-SSVPGVR polypeptide) at m/z 1342.8 in the bottom spectrum shows that the modification was quantitative. Furthermore, the reaction conditions tested did not modify arginine residues, because an additional mass shift of +160 Da at m/z 1662.9 was not observed. That GBA specifically modified the citrulline residue was also confirmed by tandem mass spectrometry (MS/MS) analysis, proving that GBA synthesized by the protocol described above can be used to specifically modify citrulline residues.

### Example 2

### Enrichment of Citrullinated Polypeptides by Soluble Biotinylated Derivatives of Glyoxalbenzoic Acid

### Preparation of a Biotinylated Phenylglyoxal Derivative

In order to combine the specific modification of citrulline with the enrichment of citrullinated peptides from heterogeneous mixtures, several biotinylated glyoxal derivatives will be synthesized that are not commercially available. For example, a biotin-lysine-phenylglyoxal derivative is synthesized by conventional solid phase chemistry on a robot used for solid phase peptide chemistry. First, Fmoc-Lys(Dde)-OH is coupled to 2-chlorotrityl resin. Next, the Fmoc group is deprotected and GBA is coupled to the α-amino group of lysine. Then the Dde protection group is cleaved by hydrazine, and biotin is coupled to the ε-amino group of lysine. Finally, the complete biotin-lysine-phenylglyoxal derivative is cleaved off the resin with acid and analyzed by HPLC and MS. This synthesis scheme is used to attach a large number of different compounds to the ε-amino group of lysine, enabling the specific modification of citrulline with virtually any tag, including fluorescent markers, peptide tags, and the like, thereby facilitating the purification, identification, and visualization of citrullinated polypeptides.

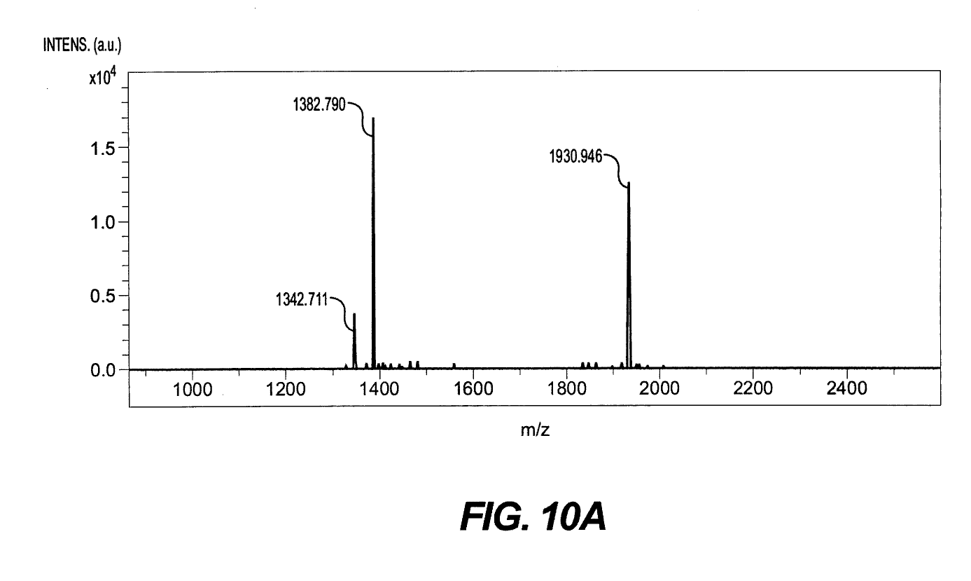

GBA was derivatized with biotin using Biotin-PEG NovaTag® resin (0.48 mmol/g binding capacity) by standard solid phase chemistry and diisopropylcarbodiimide (DIC) as a coupling reagent. Biotin NovaTag® resin may also be used (both resins were purchased from Novabiochem, Merck Biosciences, Ltd., UK). First, 5 mg resin (2.4 mmole binding capacity) was swelled and washed with dimethylformamide (DMF). Next, 1.4 mg (7.8 mmol) GBA was dissolved in DMF and 40 μmol DIC was added. The resin was incubated with this reaction mixture for 2 hours at room temperature with gentle swirling. After extensive washing, the biotinylated GBA was cleaved from the resin with a mixture of 98% TFA/2% water, producing the compound shown in FIG. 9A.

In an alternative method, Biotin-PEG-GBA was synthesized as follows. 10 mg biotin-PEG-amine (Pierce Biotechnology, Rockford, Ill., USA) were dissolved in 50 μL DMF (Fluka, St. Louis, Mo., USA) and added 19 mg GBA dissolved in 50 μL DMF. Subsequently, 13.4 mg (16.4 μL) diisopropylcarbodiimide (DIC) was added and the solution was incubated for 3 hours at 25° C.

The reaction solution was centrifuged and the supernatant was collected and diluted to 5 mL using H₂O containing 0.1% formic acid (Fluka, St. Louis, Mo., USA). This solution was first semi-purified using a C₁₈ Bond Elute (Varian, Palo Alto, Calif., USA) solid phase extraction column. The fraction containing the biotin-PEG-GBA was then purified using a series 1100 HPLC instrument (Agilent, Palo Alto, Calif., USA) equipped with a fraction collector. The identity of the purified compound (FIG. 9B) was verified using a microTOF-Q mass spectrometer from Bruker Daltonics (Bremen, Germany).

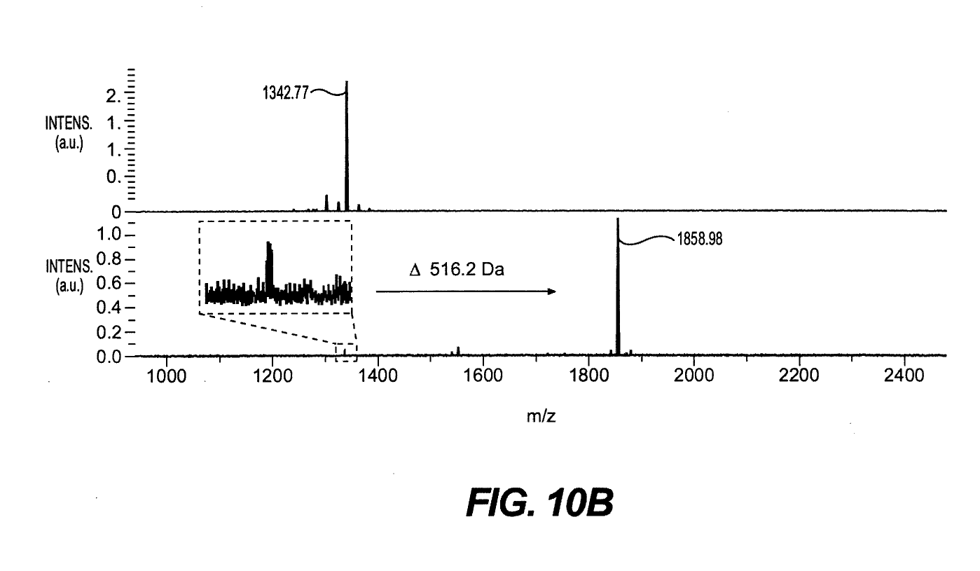

The Biotin-PEG-GBA obtained from the first procedure was used to modify a simple mixture containing the peptides SAVRA-Cit-SSVPGVR and SAVRL-R-SSVPGVR. Briefly, 5 μl of 0.4 mM SAVRA-Cit-SSVPGVR and 0.8 mM SAVRL-R-SSVPGVR was incubated with 5 μl of 98% TFA containing approximately 45 mM biotinylated GBA for two hours at 37° C. The supernatant was analyzed by MALDI-TOF MS before and after incubation. The obtained spectrum clearly shows that the SAVRA-Cit-SSVPGVR was modified by the Biotin-PEG-GBA, indicated by an increase of +558 Da in mass. See FIG. 10A.

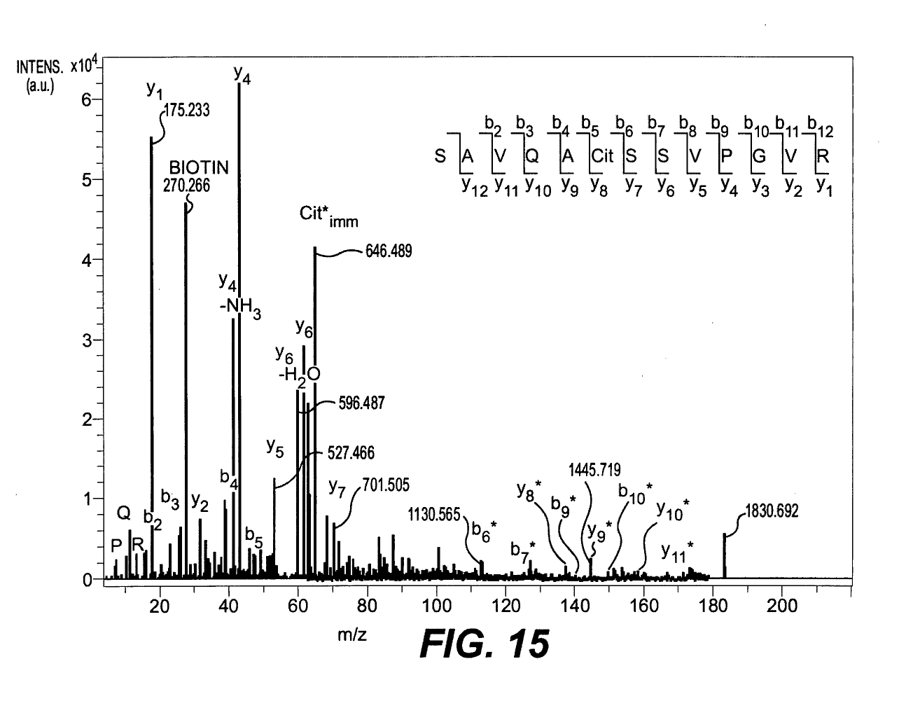

The Biotin-PEG-GBA obtained using the alternative procedure was used to modify a mixture containing the peptides SAVRA-Cit-SSVPGVR and SAVRL-R-SSVPGVR as described above. The supernatant was analyzed by MALDI-TOF MS before and after incubation. The obtained spectra clearly shows that the SAVRA-Cit-SSVPGVR was modified by the Biotin-PEG-GBA, indicated by an increase of +516.2 Da in mass. See FIG. 10B. A sample of SAVRA-Cit-SSVPGVR modified by Biotin-PEG-GBA was analyzed by MALDI TOF/TOF MS to determine the peptide sequence. FIG. 15 demonstrates that the position of the citrulline residue can be readily identified. Fragments denoted by b₂₋₁₂ indicate those not having the C-terminal R residue. Fragments denoted by y₁₋₁₂ indicate those not having the N-terminal S residue. Starred peaks indicate fragments that are modified by Biotin-PEG-GBA. Cit*ᵢₘₘ refers to the citrulline immonium ion which is modified in its side chain by Biotin-PEG-GBA.

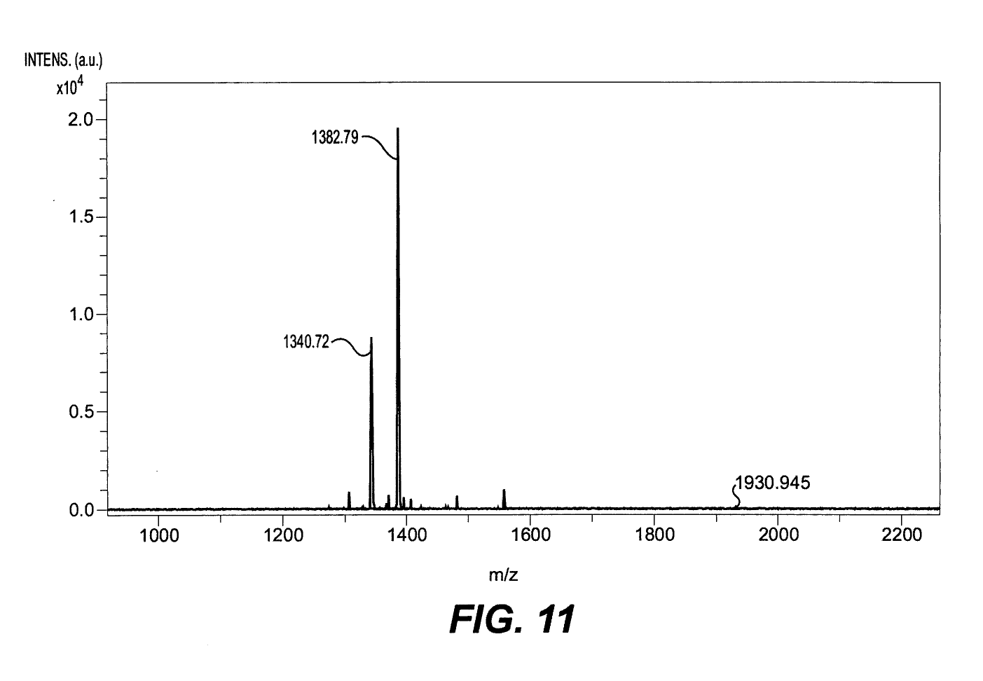

Quantitative modification of SAVRA-Cit-SSVPGVR was not obtained in this preliminary experiment because of sub-optimal reaction conditions. Tandem mass spectrometry clearly pinpointed the modification to the citrulline residue. Subsequently, the peptide mixture was purified with a combination of strong cation exchange and reversed phase (C₁₈) solid phase extraction. The purified peptide mixture was stored in PBS and applied to a micro-column of monomeric avidin beads (Pierce, Rockford, Ill., USA). MALDI-TOF MS analysis of the column flow-through showed that the modified SAVRA-Cit-SSVPGVR peptide is completely retained by the monomeric avidin particles, while the SAVRL-R-SSVPGVR peptide is not. See FIG. 11. The modified SAVRA-Cit-SSVPGVR peptide is released from the beads using a solution containing 2 mM biotin in PBS. These and other biotinylated mono- and disubstituted glyoxal derivatives are used for modification of citrullinated proteins and peptides in solution followed by their enrichment by avidin/streptavidin.

### Example 3

### Enrichment of Citrullinated Polypeptides by Immobilized GBA

### Preparation of Beads Carrying Immobilized GBA

GBA was coupled to TentaGel S HMB resin using 4-dimethylaminopyridine (DMAP) (purchased from Sigma-Aldrich, St. Louis, Mo.) as catalyst. The TentaGel S HMB resin carries a hydroxymethyl benzoic acid (HMB) at the surface. GBA is coupled to the HMB group by its carboxyl group. The resulting ester bond creates a base labile cleavage site between HMB and GBA. After the reaction of peptide-bound citrulline residues with the glyoxal moiety, immobilized peptides are released by applying basic conditions.

First, the beads were swelled in dimethylformamide (DMF) for 30 minutes, then washed 3× with dichloromethane (DCM) and 3× with DMF. Next, 0.7 mg (4 μmol) of GBA was coupled to 5 mg of TentaGel S HMB resin (binding capacity: 0.29 mmol/g; purchased from Rapp Polymere, Tübingen, Germany), using 8 μmol diisopropylcarbodiimide (DIC) as coupling reagent and 0.4 mol DMAP as catalyst for the esterification reaction. Coupling was performed for 2.5 hours at room temperature with gentle swirling. Subsequently the resins were extensively washed with DMF, methanol and diethylether before they were air dried. The resulting ester bond is stable under the highly acidic modification conditions. The extent of resin loading is examined by measuring the remaining free hydroxyl groups on the resin by routine experimentation.

### Enrichment of Citrullinated Polypeptides by Immobilized GBA

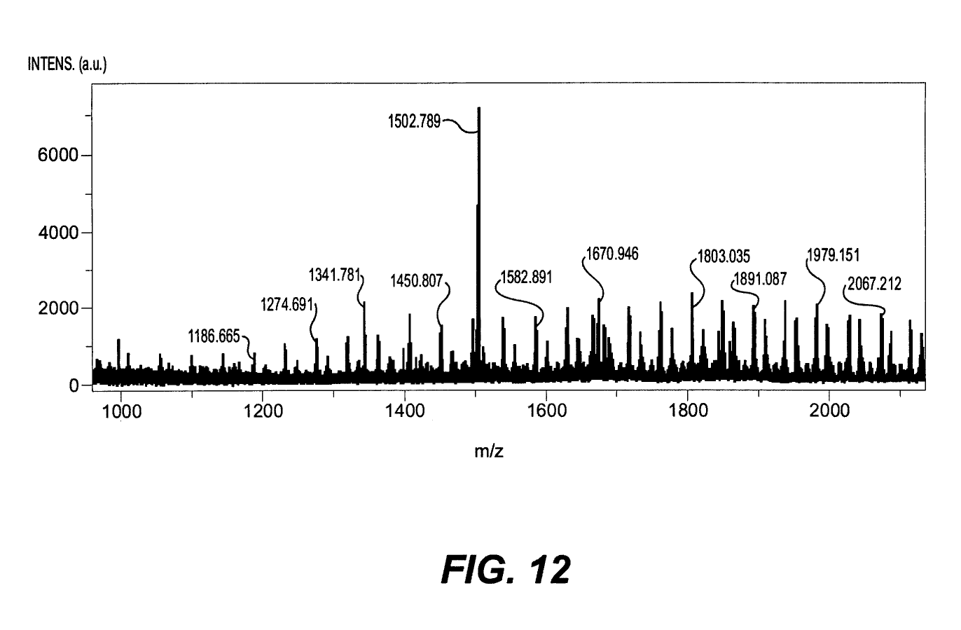

Peptides SAVRA-Cit-SSVPGVR (30 nmol) and SAVRL-R-SSVPGVR (10 nmol) were dissolved in 40 μl of 50% TFAlwater and the mixture was added to 0.1 mg of GBA-HMB-resin corresponding to 30 nmol of immobilized GBA. The sample was incubated at 45° C. for 4 hours. Aliquots of the supernatant were removed after 2 and 4 hours, then desalted and analyzed by MALDI-TOF MS (data not shown). After 4 hours, the resin was washed extensively (3× with 30 μL water, 6× with 30 μL methanol, 5× with 30 μL DCM, and 3× with 30 μL diethylether) and air dried. The resin was allowed to swell again by adding water for 30 minutes. To cleave citrullinated peptides covalently bound to the resin after reacting with the glyoxal moiety, the resin was treated with 0.3 M NaOH. This strong base cleaves the ester bond between the HMB linker and GBA. After neutralization, an aliquot of the supernatant was desalted and analyzed by MALDI-TOF MS. See FIG. 12.

The aliquots of supernatant removed from the reaction at 2 and 4 hours showed a clear signal for SAVRL-R-SSVPGVR and only a weak signal for SAVRA-Cit-SSVPGVR (data not shown). In contrast, the aliquot of supernatant containing polypeptides cleaved from the GBA-HMB resin showed a clear signal for the citrulline-containing polypeptide which was, as expected, modified with GBA. No signal was found for the non-citrulline containing peptide in that supernatant, again emphasizing the specificity of the reaction and the successful enrichment. Because we used a polyethylene glycol (“PEG”) grafted copolymer as the resin, we also unexpectedly observed a series of signals typical of PEG polymers. See FIG. 12. The protocol and type of resin used in this application will therefore be optimized in the future.

This approach will be applied to more heterogeneous mixtures containing small amounts of citrullinated polypeptides and an excess of non-citrullinated polypeptides. The incubation conditions will be optimized to achieve maximal modification and immobilization, efficient removal of unmodified polypeptides, and to explore the yield and specificity of the enrichment. In addition, this approach will be extended to citrullinated proteins. Working on the level of proteins would reduce the sample heterogeneity compared to working on the level of polypeptides, such as after digesting a protein mixture with trypsin.

### Example 4

### Determination of Citrullinated Proteins in a Biopsy of Rheumatoid Arthritis Patients

Tissue obtained from the biopsy of an inflamed joint of a patient suffering from rheumatoid arthritis is homogenized using a Waring blender and a suitable buffer containing an appropriate cocktail of protease inhibitors. The homogenate is then transferred to a glass beaker and stirred for 30-60 minutes at 4° C. Cell debris and other particulate matter are removed by centrifugation at 10,000×G for 10-20 minutes at 4° C. Floating fatty material is removed by filtration. The filtrate is then purified by reversed phase (RP) and strong cation exchange (SCX) solid phase extraction to obtain a protein fraction. Additional fractionation steps based on size, charge, or other physical or chemical characteristics are performed as necessary to reduce the complexity of the protein mixture.

Subsequently, citrullinated proteins in the different fractions are modified with biotinylated GBA or other mono- or disubstituted glyoxal derivatives according to the protocols described above, and biotinylated or otherwise tagged proteins are isolated. This step is expected to dramatically reduce the sample's complexity. Thus, proteins will be digested by trypsin or other proteases commonly used for such analysis, and known to one of ordinary skill in the art. Biotinylated peptides are isolated from the digestion mixture, reducing the complexity of the sample enormously. The purified biotinylated citrulline-containing peptides are then analyzed by LC-MS/MS. Alternatively, the modified protein mixtures can be separated by conventional 2D-gel electrophoresis followed by Western blotting and specific visualization of citrullinated proteins. Only modified (i.e., citrullinated) proteins are further analyzed. Citrullinated proteins and specific citrullination sites are subsequently identified by mass spectrometry.

### Example 5

### Immunohistochemical Studies on the Distribution of Citrullinated Proteins in Tissue

To deter mine the distribution of citrullinated peptides and proteins in various tissues, ultrathin slices of the tissue of interest are prepared (e.g., from the synovia of rheumatoid arthritis patients) and fixed by standard procedures. Using mono- or disubstituted glyoxal derivatives labeled with fluorescent markers, citrullinated proteins are specifically stained and analyzed by conventional fluorescence microscopy. The modification conditions are adapted as necessary to minimize acid concentrations required for specific staining while maintaining tissue integrity.

### Example 6

### Quantification of Citrullinated Proteins Using Isotope Labeled Citrulline-Reactive Mono- and Disubstituted Glyoxal Derivatives

Isotope labeling of citrullinated peptides, polypeptides or proteins is used both to identify the proteins or polypeptides present, and in parallel, to quantify the relative amounts of each in two different samples using mass spectrometry. This method requires synthesis of a light and heavy isotope-containing version of a mono- or disubstituted glyoxal derivative of formula (I). Thus two versions of a compound of formula (I) are synthesized in which part (e.g., either R₁, R₂, S, or Y) or all of the molecule comprises, for example, either ¹²C (light version) or ¹³C (heavy version) at each carbon atom. Other isotope pairs, such as ¹H and ²H, are substituted as desired, depending on the required chemistry. For example, Biotin-PEG-GBA is synthesized in two versions, such that one has ¹²C present at every carbon atom in the PEG spacer (light version), and one has ¹³C present at every carbon atom in the PEG spacer (heavy version).

Subsequently, two samples, each containing at least one citrulline-containing peptide, polypeptide, or protein, are modified using the reaction conditions described above. Sample 1 is modified with the light isotope version of the Biotin-PEG-GBA (¹²C-Biotin-PEG-GBA), and sample 2 is modified with the heavy isotope version of Biotin-PEG-GBA (¹³C-Biotin-PEG-GBA). The samples are then mixed together, and the biotinylated citrulline-containing peptides, polypeptides, or proteins are purified with streptavidin/avidin beads or on a streptavidin/avidin column. The isolated, modified citrulline-containing peptide, polypeptide, or protein mixtures are then digested with trypsin or other appropriate reagent and analyzed by mass spectrometry, such as MALDI-TOF or LC-tandem MS. Each labeled peptide is present in a light and a heavy version, differing by a given mass for each modified citrulline residue, depending on the isotopes used to synthesize the light- and heavy-isotope versions of the citrulline-reactive reagent (e.g, Biotin-PEG-GBA). Because the labeled peptides are otherwise chemically identical, the observed peak heights correspond to the relative amounts of both peptides. Thus, the MS analysis not only identifies different proteins, but determines their relative amounts in two different samples in the same experiment. This approach will, for example, be used to determine differences in citrullination of peptides, polypeptides, or proteins in samples derived from diseased subjects compared to healthy control subjects.

### Example 7

### Determination of the Spatial Distribution of Citrullinated Peptides, Polypeptides, and Proteins in Tissue by MALDI Imaging

Ultrathin sections of the tissue of interest are modified using a citrulline-reactive compound of formula (I), wherein the physical or molecular tag Y is a compound which produces a characteristic “signature ion” on fragmentation in a MALDI MS instrument such as, for example, a peptide sequence containing a proline residue, which is particularly prone to fragmentation. After modification with the citrulline-reactive compound, the tissue is sprayed with a matrix, such as α-CHCA or dihydroxybenzoic acid (DHB), and analyzed by MALDI TOF/TOF mass spectroscopy. By tracing the presence and intensity of the signature ion, the location of modified citrulline-containing peptides, polypeptides, or proteins can be determined. In addition, the TOF/TOF spectrum can provide some structural information regarding the identity of the modified peptides, polypeptides, or proteins. Reaction conditions are optimized to maintain integrity of the tissue being analyzed.

### Example 8

### Specific Detection of Citrullinated Proteins in a Gel or on a Western Blot

Citrulline-containing proteins in a sample are modified with a citrulline-reactive compound of formula (I), wherein the molecular tag Y is, for example, a suitable fluorophore. The sample containing modified and unmodified proteins is separated, for example, on a polyacrylamide gel (e.g., an SDS-PAGE gel or a 2D-gel of a percentage suitable for the separation of proteins in the desired size range). After thoroughly washing and/or drying the gel, modified citrulline-containing proteins are identified by standard laboratory methods for detecting, measuring, or quantifying fluorescence, such as fluorescence spectroscopy, densitometry, or the like. This approach can be combined with “differential gel electrophoresis” to determine the relative abundance of citrullinated proteins in two different samples (e.g., healthy vs. diseased). Here, the citrullinated proteins in the two samples are modified with two different citrulline-reactive compounds of formula (I) which differ only in their fluorophore Y. Sample 1 is modified with the citrulline-reactive compound containing the fluorophore Y₁, while Sample 2 is modified with the citrulline-reactive compound containing the fluorophore Y₂. Subsequently, the samples are mixed and separated on a 2D gel. The gel is read by a fluorescence scanner and the intensities obtained for the two different fluorophores report the relative abundance of citrullinated proteins in both samples investigated.

Alternatively, proteins in a sample of interested are separated on an SDS-PAGE gel of a suitable percentage as described above, and then transferred to a suitable membrane by standard methods. The citrulline-containing proteins on the membrane are then modified with a citrulline-reactive compound of formula (I), wherein the physical or molecular tag Y is a suitable fluorophore. After thoroughly washing the membrane, the size and relative amount of citrulline-containing proteins is determined by standard laboratory methods for detecting, measuring, or quantifying fluorescence, such as fluorescence spectroscopy, densitometry, or the like.

### Example 9

### Detection of Citrullinated Protein in a Mixture of Proteins by Western Blotting

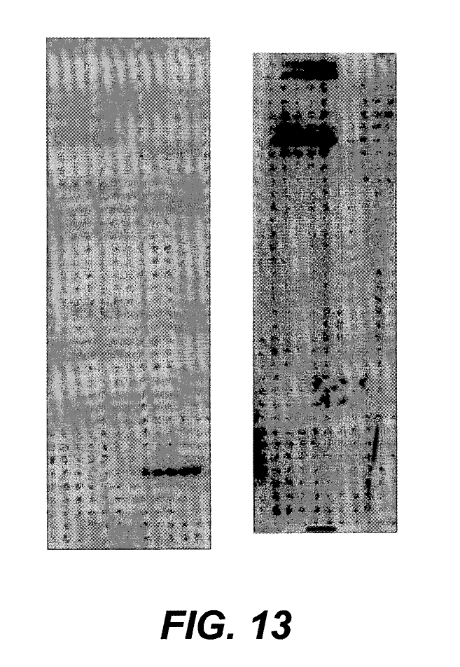

Bovine Serum Albumin (BSA) (10 μg) was citrullinated by human PAD4 enzyme that had been recombinantly expressed. See, K. Nakashima et al, J. Biol. Chem. 274, (1999) 27786-27792. Citrullination was performed by incubating 10 μg BSA with PAD in 100 mM Tris-HCl pH 7.5 containing 10 mM CaCl₂. Citrullinated BSA (3.3 μg) was combined with 3.3 μg lysozyme (14 kD) and 3.3 μg ovalbumin (43 kD) in phosphate buffered saline (PBS). Two aliquots of this mixture were concurrently separated using SDS-PAGE polyacrylimde gel electrophoresis. As shown in FIG. 13, the left gel was stained with Coomassie while the duplicate gel was blotted onto a polyvinylidene difluoride (PVDF) membrane. The membrane was blocked using 0.1% ovalbumin in PBS. The blotted proteins were crosslinked using 4% paraformaldehyde.

The membrane was placed in a solution of Biotin-PEG-GBA (prepared according to the alternative procedure of Example 2) in 50% TFA and held at 37° C. for approximately 16 hours. The membrane was then incubated with streptavidin-horseradish peroxidase conjugate (streptavidin-HRP) diluted 1:10000 in Tris buffered saline containing 0.1% Tween 20 (TBS-T) containing 1% BSA. Then the blot was washed with TBS-T. The blot was developed using an ECL Western Blot Detection Kit from Amersham Biosciences (Pittsburgh, Pa., USA).

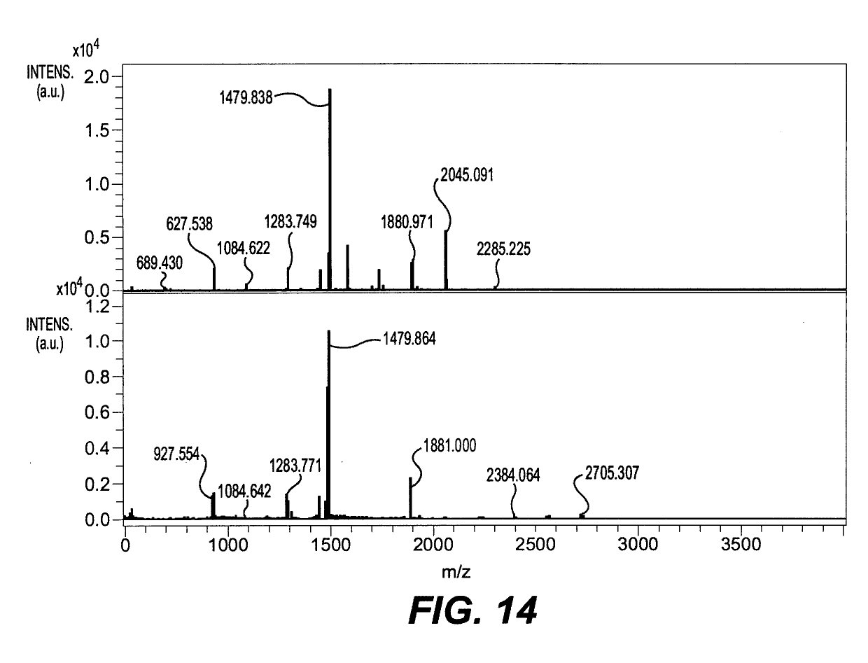

As shown in FIG. 13, the developed Western Blot (right) had a lower band that was identified as citrullinated BSA. Two higher molecular weight bands were identified as BSA contaminants, such that they were also citrullinated by PAD4. As shown in FIG. 14, the lower band (top) and the upper bands (bottom) on the Western blot were analyzed by MALDI TOF-MS spectroscopy. The blot did contain show any bands corresponding to lysozyme or ovalbumin. Thus, Biotin-PEG-GBA is useful to visualize deiminated proteins after Western blotting.

### Example 10

### Purification of a Citrullinated Peptide Mixture Modified by Biotin-PEG-GBA

A citrullinated peptide mixture is modified by Biotin-PEG-GBA according to the procedure of Example 2. After modification, the sample is dried, then reconstituted in SCX binding/washing solution which contains 50% acetonitrile (ACN) (Rathburn, Walkerburn, Scotland) and 50% 0.1% formic acid (FA) (Fluka, St. Louis, Mo., USA), in water. The solution is passed through a column of SCX polysulfoethyl aspartamide beads obtained from PolyLC (Columbia, Md., USA). The peptides bind to the SCX beads while excess Biotin-PEG-GBA passes through the column. The SCX beads are washed with SCX binding/washing solution. Then, SCX elution solution (25% acetonitrile and 75% 1M NaCl (Fluka, St. Louis, Mo., USA)) in water is used to elute the bound peptides.

Enrichment of citrullinated peptides can be performed using a biotin-streptavidin binding process. The eluent containing the previously bound peptides is added to 0.1% SDS to prevent unspecific binding. The solution is incubated with Dynabeads® M-270 Streptavidin (Invitrogen, Carlsbad, Calif.) for approximately 20 min. The supernatent is removed and unspecifically bound peptides are washed off of the beads using PBS containing 0.1% SDS. Washing with ACN/1M NaCl (25/75, v/v) also removes unspecifically bound peptides and residual SDS. Residual NaCl is removed with ACN/H₂O (20/80, v/v). Then, modified citrullinated peptides are eluted with streptavidin elution solution (ACN/10% FA/2 mM biotin (70/10/20, v/v)). Biotin was purchased from Sigma (St. Louis, Mo., USA). The solution of citrullinated peptides can be analyzed by, for example, MALDI-TOF MS.

### Example 11

### Enrichment of Citrullinated Peptides from Synovial Fluid

Citrullinated peptides are known to be present in synovial fluid. Synovial fluid proteins (280 μg) were obtained from a patient having rheumatoid arthritis. The proteins were separated using SDS-PAGE polyacrylimide gel electrophoresis. The resulting gel lane was sliced into eight equally sized sections. The proteins were reduced using 10 mM DTT in 100 mM ammonium bicarbonate pH 8.4. Both reagents were from Sigma (St. Louis, Mo., USA). The samples were incubated at 56° C. for 1 hour. Then the proteins were alkylated using 50 mM iodoacetamide (Sigma, St. Louis, Mo., USA) in 100 mM ammonium bicarbonate pH 8.4. The samples were incubated in the dark at room temperature for 45 minutes. The proteins were then digested by adding 50 μL 10 ng/μL trypsin (Promega, Madison, Wis., USA) in 50 mM ammonium bicarbonate. Digestion was performed at 37° C. for 16 hours.

A-Cit-SSVPGVR (500 fmol) was added to the resulting extracted peptide solution as a positive-control reference peptide. The peptides were modified using Biotin-PEG-GBA according to the alternative procedure of Example 2. The modified peptide mixture was purified using the SCX procedure of Example 10 to remove excess Biotin-PEG-GBA. Then, the citrullinated peptides were enriched using the Dynabeads® M-270 Streptavidin procedure of Example 10.

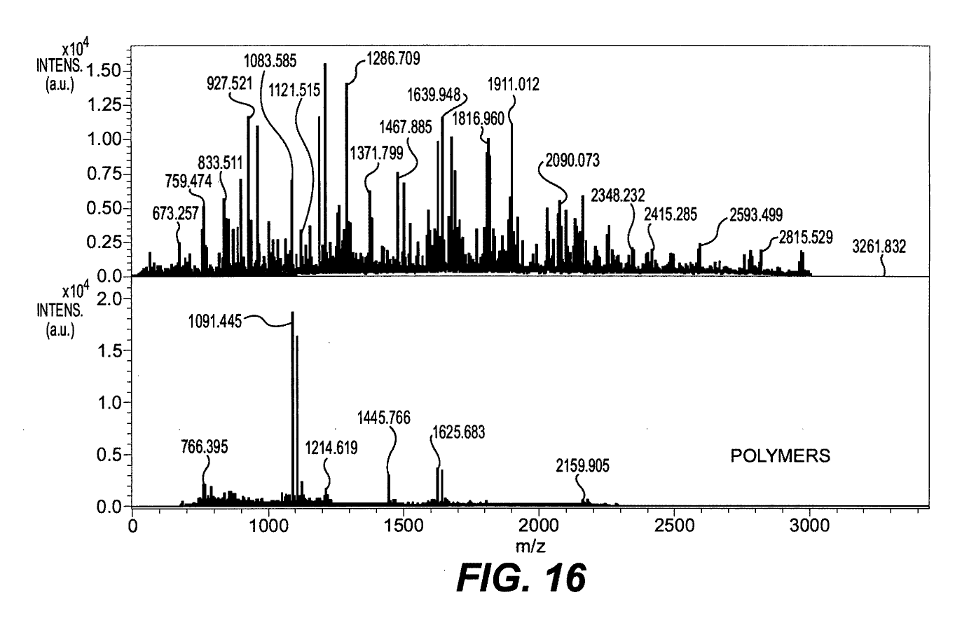

A sample of Gel section 4 prior to modification and its streptavidin eluate were analyzed by MALDI-TOF MS, as shown in FIG. 16. The top spectrum of gel shows the range of peptides and proteins present in synovial fluid from Gel section 4. However, none of these were citrullinated. The only peptide in the streptavidin eluate (FIG. 16 bottom) was the positive control A-Cit-SSVPGVR appearing at 1445.766 m/z, with a few Biotin-PEG-GBA polymers which are highlighted on the spectrum. This study shows the specificity of the method for identifying citrullinated peptides. The amount of the positive control was 12.5 fmol, demonstrating the high degree of sensitivity of the method.

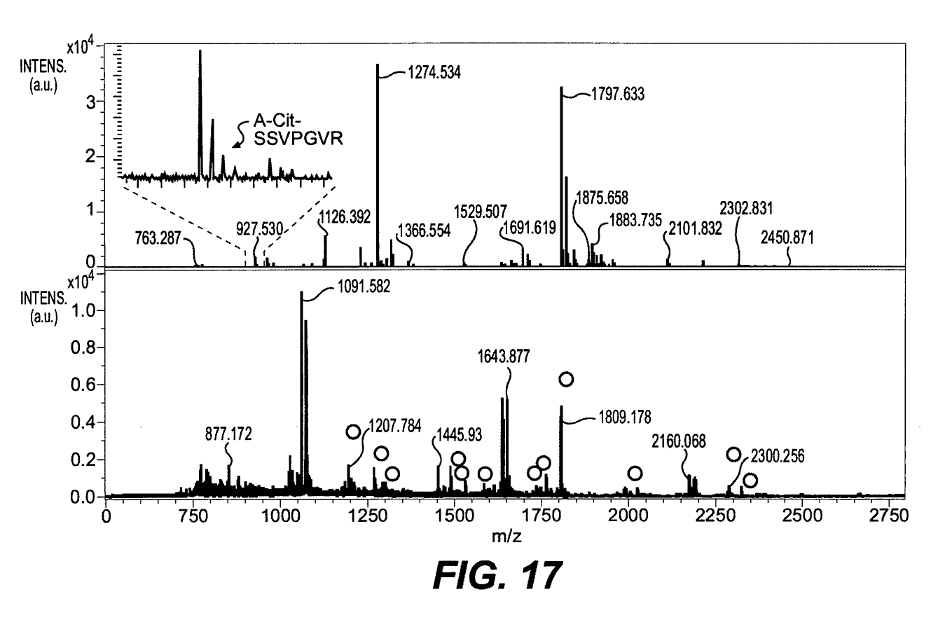

As shown in FIG. 17, samples of Gel section 8 prior to modification (top) and its streptavidin eluate (bottom) were analyzed by MALDI-TOF MS. Gel section 8 differs from that of Gel section 4 in that Gel section 8 contained citrullinated peptides. The streptavidin eluate contained a mixture of peptides that had been enriched in those having citrulline residues. The positive control A-Cit-SSVPGVR appears at 1445.935 m/z. Marked peaks indicate those having citrulline residue(s).

These results show how this process can effectively separate citrullinated proteins from a complex mixture of synovial fluid proteins. The detection limit of citrullinated peptides is below 10 fmol, demonstrating excellent sensitivity. The streptavidin eluates can also be analyzed by MALDI TOF/TOF MS to determine the peptide sequence as described in Example 2.

### Example 12

### Detection of Citrullinated Peptides by Reaction with Immobilized GBA

A BSA tryptic digest was obtained by deimination of BSA by peptidylarginine deiminase-type 4. A reaction mixture containing 5 μg human peptidylarginine deiminase, 60 μg BSA, 0.2 M Tris-HCl, pH 7.5, 10 mM DTT and 10 mM CaCl₂ was gently mixed in an 1.5 ml Eppendorf cup. The mixture was left for incubation at 37° C. overnight. An aliquot was subjected to 0.05 μg trypsin (produsent) in 50 mM ammonium bicarbonate for digestion over night at 37° C.

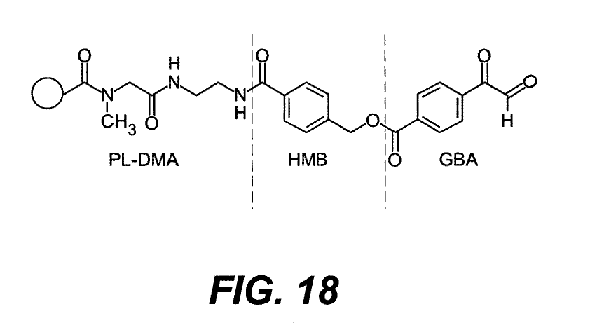

GBA was immobilized on PL-DMA beads (Polymerlabs) using HMB (hydroxymethylbenzoic acid) as a base cleavable linker, whose structure is shown in FIG. 18. The PL-DMA/HMB/GBA resin was incubated at a low pH (50% TFA in water, for 1-2 hours at 45° C.) with an aliquot of the tryptic digest. Peptides having citrulline residues covalently bound to the GBA immobilized on the resin. Unbound peptides were washed away using washing solutions of decreasing acidity; 50% TFA in DMF (4 times), 25% TFA in DMF (twice), 5% TFA in DMF (twice) and at last in water (5 times). Bound peptides were released from the resin at the HMB linker by incubation for 20 minutes with 0.2M NaOH (VWR, West Chester, Pa., USA) in water containing guanidine (Sigma, St. Louis, Mo., USA) in equimolar amounts compared to the amount of GBA present on the beads.

The resulting released peptides were those modified by GBA. The peptides were desalted using a RP C₁₈ microcolumn prior to analysis by MALDI-TOF MS. GBA-modified citrullinated peptides show a mass increase of 160 Da.

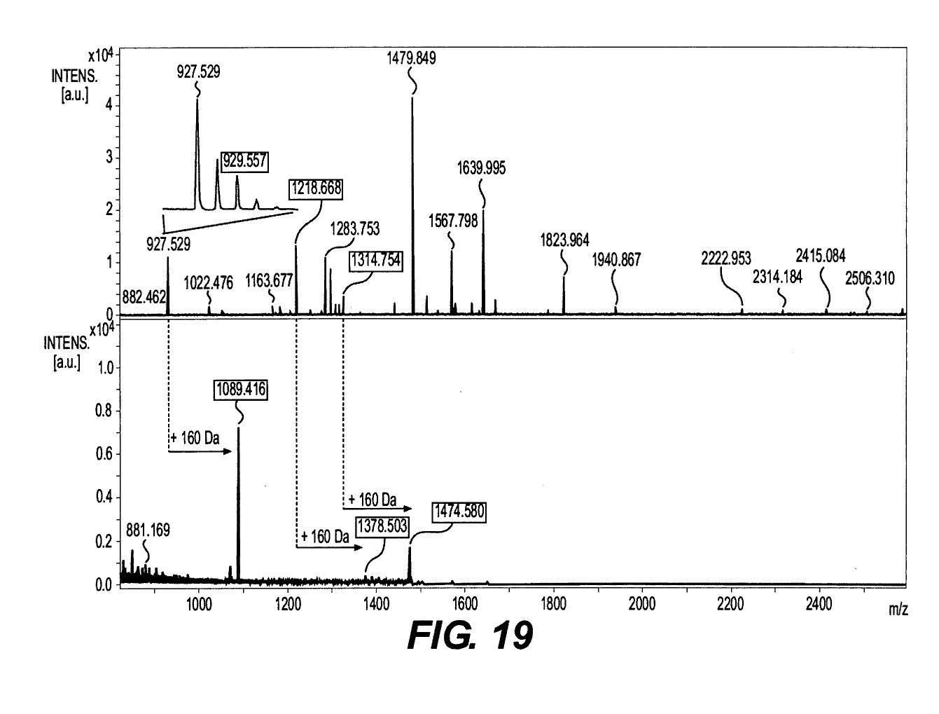

FIG. 19 shows a comparison of a MALDI-TOF MS spectrum of peptides from a BSA digest having citrulline-containing peptides added prior to incubation with a PL-DMA/HMB/GBA resin (top) with that of the peptides modified by GBA that were released from the resin (bottom). The highlighted peaks indicate that the released peptides are only those containing citrulline that have been modified by GBA.

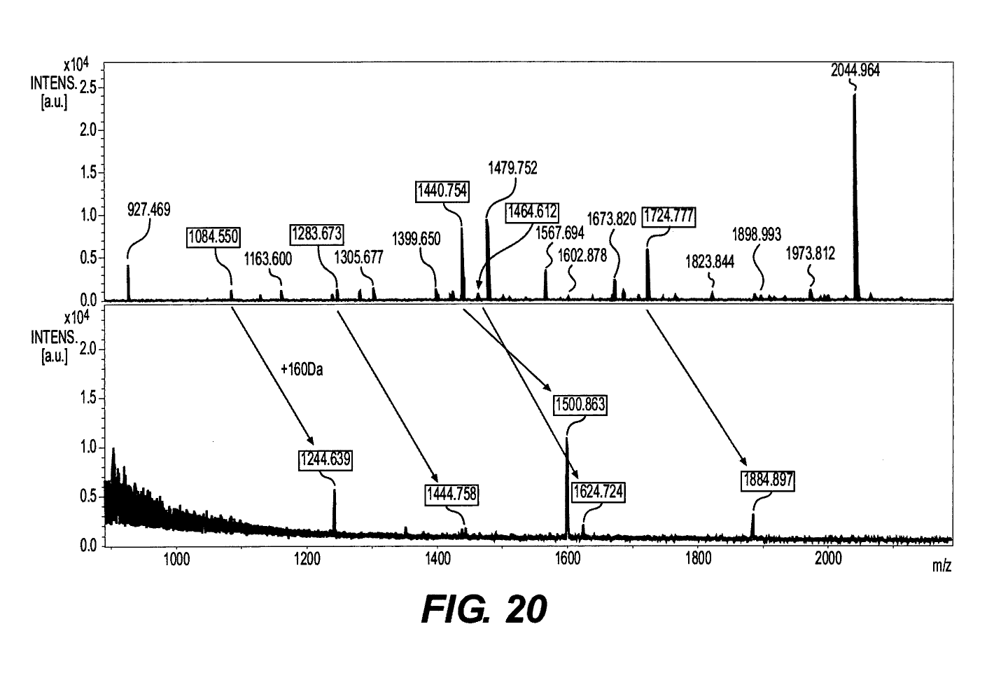

FIG. 20 provides a comparison of a MALDI-TOF MS spectrum of peptides from a deiminated BSA tryptic digest prior to incubation with a PL-DMA/HMB/GBA resin (top) with that of peptides modified by GBA that were released from the resin (bottom). The known citrulline peptides in the tryptic digest are highlighted to demonstrate how only those peptides remained in the released GBA-modified peptide mixture. Citrullinated peptides were confirmed by MALDI TOF/TOF MS.

These comparisons demonstrate how citrulline-containing peptides can be specifically enriched from highly heterogeneous peptide mixtures.

### Example 13

### Modification of a Homocitrulline-Containing Peptide with Biotin-PEG-GBA

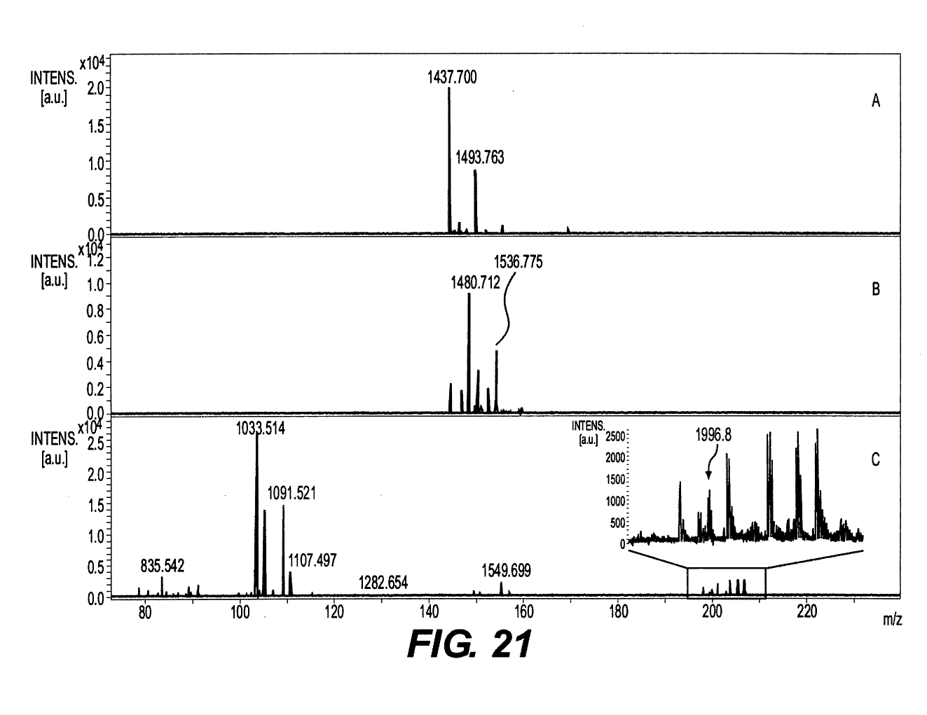

Unpurified peptide Ac-FWADKEEEWR (1 pmol) was carbamylated using 2M potassium cyanate with incubation at 37° C. for 6 hours. FIG. 21A shows the peptide prior to carbamylation at 1437.64 m/z, with a peak at 1493.76 m/zfor a t-butyl protected derivative. FIG. 21B shows the peptide after carbamylation, with peaks shifted by +43 Da. The carbamylated peptide was desalted but not further purified prior to modification using Biotin-PEG-GBA (3.5 hours at 37° C. using the alternative procedure of Example 2). The resulting modified peptide, containing some excess Biotin-PEG-GBA, was dried under vacuum and redissolved in acetonitrile/0.1% formic acid. Modification increased the mass of the peptide by 516.2 Da such that the modified peptide is observed at 1996.8 m/z, as shown in FIG. 21C. Several peptide impurities containing homocitrulline are also observed in the range of the modified peptide. These results demonstrate that homocitrulline can be modified using the Biotin-PEG-GBA procedures described for citrulline and confirms their similar chemical reactivity. It is expected that the enrichment methods exemplified for citrulline-containing peptides will be equally effective with peptides comprising homocitrulline.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### LITERATURE CITED

Arita, K. et al., “Structural basis for histone N-terminal recognition by human peptidylarginine deiminase 4,” PROC. NATL. ACAD. SCI. U.S.A. 103(14):5291-5296 (2006).
György, B. et al., “Citrullination: A posttranslational modification in health and disease,” INT'L J. BIOCHEM. CELL BIOL. 38:1662-77 (2006).
Vossenaar, E. R., et al., “PAD, a growing family of citrullinating enzymes: genes, features and involvement in disease,” BIOESSAYS 25:1106-1118 (2003).
Wang, Z. et al., “Protein Carbamylation links inflammation, smoking, uremia and atherogenesis,” NATURE MEDICINE 13:1176-1184 (2007).

1. A compound of formula (I): 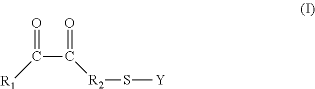
wherein R₁ and R₂ are chosen from any branched or unbranched alkyl or aryl chain of different size, length, hydrophobicity, water-solubility, positive or negative inductive effect, positive or negative mesomeric effect, or a hydrogen; wherein S is a spacer chosen from any branched or unbranched aliphatic or polyethylene glycol-based chain of variable size, length, and hydrophobicity; and wherein Y is a physical or molecular tag that facilitates identification, visualization, detection or purification of citrulline and/or homocitrulline-containing peptides, polypeptides or proteins labeled with the compound of formula (I).

2-64. (canceled)

65. The compound according to claim 1, wherein R₁ is chosen from —H, —CH₃, and a phenyl group.

66. The compound according to claim 1, wherein R₂ is chosen from —H, —CH₃, and a phenyl group.

67. The compound according to claim 1, wherein S is —(CH₂)ₙ—or —(O—CH₂—CH₂)ₙ—, and n=1 to 25.

68. The compound according to claim 67, wherein S further comprises a cleavage site, a disulfide bond, a photocleavable group, a base labile group, or an enzymatic cleavage site.

69. The compound according to claim 68, wherein the photocleavable group is o-nitrobenzyl or pivaloyl.

70. The compound according to claim 68, wherein the enzymatic cleavage site is chosen from trypsin, chymotrypsin, Lys-C, Asp-N, and Glu-C.

71. The compound according to claim 1, wherein Y is chosen from a magnetic bead, a resin, and a solid support.

72. The compound according to claim 1, wherein Y is chosen from biotin, iminobiotin, biotinyl-6-aminohexanoic acid, a His-tag, a metal affinity tag (SEQ ID NO:1), a FLAG peptide (SEQ ID NO:2), digoxin, a dinitrophenyl group, a nitrotyrosine residue, fluorescein isothiocyanate, Texas Red, and rhodamine.

73. A method of modifying citrulline and/or homocitrulline-containing peptides, polypeptides, or proteins under acidic conditions comprising:
contacting a solution or sample comprising at least one citrulline and/or homocitrulline-containing peptide, polypeptide or protein, and
at least one citrulline and/or homocitrulline-reactive compound according to claim 1,
wherein the citrulline and/or homocitrulline-reactive compound becomes covalently attached to at least one citrulline residue within the at least one citrulline and/or homocitrulline-containing peptide, polypeptide, or protein in the solution or sample.

74. A method of isolating, enriching or purifying citrulline and/or homocitrulline-containing peptides, polypeptides, or proteins from a solution or sample, comprising:
a) contacting at least one citrulline and/or homocitrulline-reactive compound according to claim 1, with a solution or sample comprising at least one citrulline and/or homocitrulline-containing peptide, polypeptide, or protein; wherein the at least one citrulline and/or homocitrulline-reactive compound becomes covalently attached to at least one citrulline and/or homocitrulline residue within the at least one citrulline and/or homocitrulline-containing peptide, polypeptide, or protein in the solution or sample;
b) collecting the modified citrulline and/or homocitrulline-containing peptides, polypeptides, or proteins; and
c) removing the unmodified peptides, polypeptides, or proteins from the solution or sample.

75. The method according to claim 74, wherein the citrulline and/or homocitrulline-reactive compound is Biotin-PEG-4-glyoxalbenzoic acid (GBA).

76. A method of isolating, enriching, or purifying citrulline and/or homocitrulline-containing peptides, polypeptides, or proteins from a solution or sample, comprising:
a) preparing a solution or sample comprising at least one citrulline and/or homocitrulline-containing peptide, polypeptide, or protein;
b) contacting the solution or sample with resin or beads under acidic conditions, wherein the resin or beads comprise a citrulline and/or homocitrulline-reactive group linked to the resin or magnetic beads by a cleavable spacer;
d) allowing the citrulline and/or homocitrulline-reactive group on the resin or magnetic beads to react with and modify the at least one citrulline and/or homocitrulline-containing peptide, polypeptide, or protein in the solution or sample;
e) removing any non-citrulline and/or homocitrulline-containing peptides, polypeptides, or proteins from the solution or sample;
f) cleaving the spacer; and
g) collecting the modified at least one citrulline and/or homocitrulline-containing peptide, polypeptide, or protein.

77. The method according to claim 76, wherein the resin comprising a cleavable spacer is TentaGel S 4-hydroxymethylbenzoic acid resin.

78. The method according to claim 76, wherein the beads comprising a cleavable spacer are M-280 4-hydroxymethylbenzoic acid Dynabeads®.

79. A method of detecting or visualizing citrulline and/or homocitrulline-containing peptides, polypeptides, or proteins in a solution or sample, comprising:
a) obtaining a solution or sample comprising at least one citrulline and/or homocitrulline-containing peptide, polypeptide, or protein;
b) modifying the at least one citrulline and/or homocitrulline-containing peptide, polypeptide, or protein in the solution or sample by the method according to claim 73; and
c) detecting the at least one modified citrulline and/or homocitrulline-containing peptide, polypeptide, or protein in the solution or sample.

80. The method according to claim 79, further comprising the step of fractionating or fixing the biological sample before detecting the at least one modified citrulline and/or homocitrulline-containing peptide, polypeptide, or protein.

81. A method of quantifying relative amounts of citrulline and/or homocitrulline-containing peptides, polypeptides, or proteins in a solution or sample, comprising:
a) obtaining a first solution or sample comprising at least one citrulline and/or homocitrulline-containing peptide, polypeptide, or protein and a second solution or sample comprising at least one citrulline and/or homocitrulline-containing peptide, polypeptide, or protein;
b) modifying the at least one citrulline and/or homocitrulline-containing peptide, polypeptide, or protein in the first solution or sample with a ¹²C-labeled version of a citrulline and/or homocitrulline-reactive compound of formula (I): 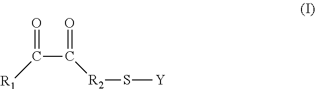
wherein R₁ and R₂ comprise any branched or unbranched alkyl or aryl chain of different size, length, hydrophobicity, water-solubility, positive or negative inductive effect, positive or negative mesomeric effect, or a hydrogen; wherein S is a spacer comprising any branched or unbranched aliphatic or polyethylene glycol-based chain of variable size, length, and hydrophobicity; and wherein Y is a physical or molecular tag that facilitates identification, visualization, detection or purification of citrulline and/or homocitrulline-containing peptides, polypeptides or proteins labeled with the compound;
c) modifying the at least one citrulline and/or homocitrulline-containing peptide, polypeptide, or protein in the second solution or sample with a ¹³C-labeled version of a citrulline and/or homocitrulline-reactive compound of formula (I);
d) mixing the first solution or sample modified with the ¹²C-labeled version of a citrulline and/or homocitrulline-reactive compound and the second solution or sample modified with the ¹³C-labeled version of a citrulline and/or homocitrulline-reactive compound of formula (I);
e) digesting the labeled mixture with trypsin;
f) collecting the modified citrulline and/or homocitrulline-containing peptides, polypeptides, or proteins from the digested, labeled mixture;
g) determining the relative amount of each citrulline and/or homocitrulline-containing peptide, polypeptide, or protein collected from the digested, labeled mixture by MALDI-TOF or LC-tandem mass spectrometry; and
h) identifying each modified peptide, polypeptide, or protein by MS/MS.

82. The method according to claim 81, wherein the citrulline and/or homocitrulline-reactive compound of formula (I) is Biotin-PEG-GBA.

83. A method of assessing the efficacy of a treatment for a disease associated with altered citrullination and/or homocitrullination of at least one protein, comprising:
a) obtaining a first biological sample from a patient suffering from the disease before treatment;
b) administering the treatment;
c) obtaining a second biological sample from said patient after treatment; and
d) determining the relative amounts of at least one citrulline and/or homocitrulline-containing peptide, polypeptide, or protein in the biological samples by the method according to claim 79;
wherein altered citrullination and/or homocitrullination of at least one peptide, polypeptide, or protein in the second biological sample relative to the first biological sample indicates the treatment is effective.

84. The method according to claim 83, wherein the disease is multiple sclerosis or rheumatoid arthritis.

85. A kit for quantifying the relative amounts of citrulline and/or homocitrulline-containing peptides, polypeptides, or proteins in a solution or sample by the method according to claim 81, comprising:
a) a light and a heavy version of a citrulline and/or homocitrulline-reactive compound of formula (I);
b) avidin/streptavidin coated magnetic beads; and
c) tubes, containers, reaction vessels, buffers, and reagents required to perform the steps of the method.

86. The kit according to claim 85, wherein the citrulline and/or homocitrulline-reactive compound of formula (I) is Biotin-PEG-GBA.

87. A kit for isolating, enriching, or purifying citrulline and/or homocitrulline-containing peptides, polypeptides, or proteins from a solution or sample by the method according to claim 74, comprising:
a) beads, resins or solid supports derivatized with a citrulline and/or homocitrulline-reactive compound of formula (I); and
b) tubes, containers, reaction vessels, buffers, and reagents required to perform the steps of the method.

88. The kit according to claim 87, wherein the citrulline and/or homocitrulline-reactive compound of formula (I) is GBA.61.

89. A kit for detecting or visualizing citrulline and/or homocitrulline-containing peptides, polypeptides, or proteins in a solution or sample by the method according to claim 79, comprising:
a) a citrulline and/or homocitrulline-reactive compound of formula (I); and
b) tubes, containers, reaction vessels, buffers, and reagents required to perform the steps of the method.

90. The kit according to claim 89, wherein the citrulline and/or homocitrulline-reactive compound of formula (I) is Biotin-PEG-GBA.

91. The kit according to claim 89, wherein the citrulline and/or homocitrulline-containing peptides, polypeptides, and proteins are visualized using streptavidin conjugated with alkaline phosphatase or horseradish peroxidase.

92. The kit according to claim 89, further comprising reagents for fractionating the solution or sample by chromatography, filtration, precipitation by salt, pH, or organic solvent, gel electrophoresis or Western blotting.

93. The kit according to claim 89, further comprising reagents for formalin fixation, paraffin embedding, and sectioning of the solution or sample.
